(19) United States
(12) Patent Application Publication
(10) Pub. No.: US 2024/0148688 A1
(43) Pub. Date: May 9, 2024

(54) METHODS FOR TREATING AND AMELIORATING CANCER

(71) Applicant: The Regents of the University of California, Oakland, CA (US)

(72) Inventors: Catriona JAMIESON, San Diego, CA (US); Leslie CREWS ROBERTSON, San Diego, CA (US); Michael BURKART, San Diego, CA (US); Larisa BALAIAN, San Diego, CA (US); Phoebe MONDALA, San Diego, CA (US); Cayla MASON, San Diego, CA (US); Raymond H. DIEP, San Diego, CA (US); James LACLAIR, San Diego, CA (US); Thomas WHISENANT, San Diego, CA (US); Warren C. CHAN, San Diego, CA (US); Inge VAN DER WERF, San Diego, CA (US); Peggy WENTWORTH, San Diego, CA (US); Wenxue MA, San Diego, CA (US)

(21) Appl. No.: 18/275,388

(22) PCT Filed: Feb. 1, 2022
(86) PCT No.: PCT/US2022/014663

§ 371 Date: Aug. 1, 2023

Related U.S. Application Data
(60) Provisional application No. 63/144,378, filed on Feb. 1, 2021.

(51) Int. Cl. A61K 31/365; A61K 31/506; A61P 35/02

(52) U.S. Cl. CPC A61K 31/365; A61K 31/506; A61P 35/02

(57) ABSTRACT

In alternative embodiments, provided are methods for treating and ameliorating a cancer, or recurrence of a cancer such as acute myeloid leukemia (AML) comprising administration to an individual in need thereof a pharmaceutical composition comprising 17S-FD-895 (or rebecsinib) and second drug such as an ATP-competitive protein tyrosine kinase inhibitor such as dasatinib. In alternative embodiments, provided are methods for the in vivo inhibition of myeloproliferative neoplasm (MPN) or AML stem cell propagation comprising administration to an individual in need thereof a pharmaceutical composition comprising 17S-FD-895 and second drug. In alternative embodiments, provided are methods for the in vivo inhibition pre-leukemia stem cell (pre-LSC) transformation into leukemia stem cells (LSCs) comprising administration to an individual in need thereof a pharmaceutical composition comprising 17S-FD-895 and second drug.

### RELATED APPLICATIONS

This Patent Convention Treaty (PCT) International Application claims the benefit of priority under 35 U.S.C. § 119(e) of U.S. Provisional Ser. Application No. (U.S. Ser. No.) 63/144,378, filed Feb. 1, 2021. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under R01DK114468 and R01CA205944 awarded by the National Institutes of Health (NIH) and 80JSC020F0200 awarded by NASA. The government has certain rights in the invention.

### TECHNICAL FIELD

This invention generally relates to medicine and pharmacology. In alternative embodiments, provided are methods for treating and ameliorating a cancer, or recurrence of a cancer, such as acute myeloid leukemia (AML) comprising administration to an individual in need thereof a pharmaceutical composition comprising 17S-FD-895 (also known as rebecsinib) and a second drug such as an ATP-competitive protein tyrosine kinase inhibitor such as fedratinib or dasatinib. In alternative embodiments, provided are methods for the in vivo inhibition of myeloproliferative neoplasm (MPN) or AML stem cell propagation comprising administration to an individual in need thereof a pharmaceutical composition comprising 17S-FD-895 (rebecsinib) and a second drug. In alternative embodiments, provided are methods for the in vivo inhibition pre-leukemia stem cell (pre-LSC) transformation into leukemia stem cells (LSCs) comprising administration to an individual in need thereof a pharmaceutical composition comprising 17S-FD-895 (rebecsinib) and a second drug.

### BACKGROUND

Cumulative evidence suggests that dormant, self-renewing cancer stem cells (CSCs), particularly in hematologic malignancies such as myeloproliferative neoplasms (MPNs), myelodysplastic syndrome (MDS), acute myeloid leukemia (AML), chronic lymphocytic leukemia (CLL) and multiple myeloma (MM), promote therapeutic resistance to chemotherapeutic agents that target dividing cells and thus promote relapse, which is the leading cause of cancer-related mortality. Relapse in these malignancies has been linked to splicing deregulation-induced clonal evolution that drives expansion of CSCs (Jiang 2017).

### SUMMARY

In alternative embodiments, provided are pharmaceutical or therapeutic compositions, formulations or therapeutic combinations of drugs comprising:
(a) rebecsinib, also called 17S-FD-895,

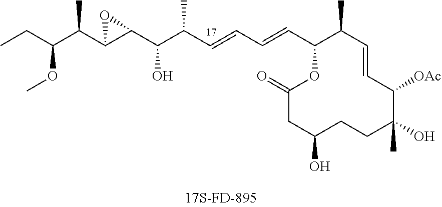 [SMILES: CC[C@H](OC)[C@@H](C)[C@H]1O[C@@H]1[C@@H](O)[C@H](C)/C=C/C=C/[C@H]1OC(=O)C[C@H](O)CC[C@@](C)(O)[C@@H](OC(C)=O)/C=C/[C@@H]1C]

or an enantiomer, stereoisomer, deuterated version, or salt thereof; and
(b) at least one second drug.

In alternative embodiments, provided are methods for:
treating and ameliorating a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM);
in vivo inhibition of myeloproliferative neoplasm (MPN) or AML stem cell propagation;
the in vivo inhibition pre-leukemia stem cell (pre-LSC) transformation into leukemia stem cells (LSCs),
the in vivo inhibition of splicesomes in AML, or MPN, and/or
the in vivo inhibition of transcript binding to a component of a spliceosome binding pocket or binding to ADAR1 (adenosine deaminase acting on RNA-1),
comprising:
administration to an individual in need thereof a formulation, a pharmaceutical composition or a therapeutic combination of drugs, comprising:
(b) rebecsinib, also called 17S-FD-895,

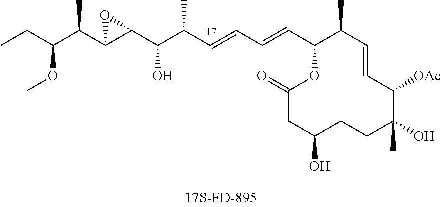 [SMILES: CC[C@H](OC)[C@@H](C)[C@H]1O[C@@H]1[C@@H](O)[C@H](C)/C=C/C=C/[C@H]1OC(=O)C[C@H](O)CC[C@@](C)(O)[C@@H](OC(C)=O)/C=C/[C@@H]1C]

or an enantiomer, stereoisomer, deuterated version, or salt thereof, or
(b) a compound of (a), or rebecsinib or 17S-FD-895, and at least one second drug.

In alternative embodiments of methods or pharmaceutical or therapeutic compositions, formulations or therapeutic combinations of drugs, as provided herein:
doses of 17S-FD-895 are administered once a day for between one to two weeks, twice a week for 2 weeks or between about one to two weeks, followed by 2 weeks rest or 2 to 4 weeks rest, with a duration of two, three, four, five or six cycles, optionally with a duration of four 28 day or monthly cycles, or the pharmaceutical or therapeutic compositions, formulations or therapeutic combinations of drugs are formulated for this dosage administration;
the at least one second drug comprises an ATP-competitive protein tyrosine kinase inhibitor, wherein optionally the ATP-competitive protein tyrosine kinase inhibitor comprises dasatinib (or SPRYCEL™or DASANIX™);
the at least one second drug comprises a JAK2 (Janus kinase 2) inhibitor, optionally fedratinib (or INREBIC™), or fedratinib and at least one second drug, wherein optionally the fedratinib is dosaged at 60 mg/kg twice daily orally, optionally for one to two or more weeks;
the at least one second drug comprises a chemotherapeutic agent, wherein optionally the chemotherapeutic agent comprises one, two, three or more of: afatinib (or GILOTRIF™), afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, fedratinib (or INREBIC™), floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib (or OPZELURA™), seliciclib, sorafenib (or NEXAVAR™), streptozocin, sulfatinib, sunitinib (or SUTENT™), tamoxifen (or NOLVADEX™), tandutinib, temozolomide, temsirolimus, teniposide, theliatinib, thioguanine, thiotepa, topotecan, uramustine, valrubicin, vandetanib, vemurafenib (or ZELBORAE™), vincristine (or ONCOVIN™), vinblastine (or VELBAN™), vinorelbine (or NAVELBINE™), and vindesine (or eldisine);
the at least one second drug comprises a hypomethylating agent (HMA), wherein optionally the HMA comprises azacitidine (or VIDAZA™) or decitabine (or DACOGEN™);
the at least one second drug comprises a second telomerase inhibitor, wherein optionally the telomerase inhibitor comprises at least one, two or three of: imetelstat, zidovudine (or azidothymidine (AZT)), stavudine (or ZERIT™), tenofovir or tenofovir disoproxil (or VIREAD™), didanosine (or VIDEX™), abacavir (ZIAGEN™), TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, ribovax, Vbx-016, ZI-HX, ZI-H04, and ZIH-03;
the formulation, pharmaceutical composition or therapeutic combination of drugs or an active agent or drug contained therein is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen;
an active agent used in methods as provided herein, or a drug in the formulation, pharmaceutical composition or therapeutic combination of drugs as provided herein, is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more;
an active agent used in methods as provided herein, or a drug in the formulation, pharmaceutical composition or therapeutic combination of drugs as provided herein, is administered as or formulated with or formulated as an) inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially;
an active agent used in methods as provided herein, or a drug in the formulation, pharmaceutical composition or therapeutic combination of drugs as provided herein, is or are administered to an individual in need thereof: using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or intravenously (IV) or intramuscularly (IM).

In alternative embodiments, provided are pharmaceutical compositions, formulations or therapeutic combinations comprising 17S-FD-895 (or rebecsinib), or 17S-FD-895 and at least one second drug, for use in:
treating and ameliorating a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM);
in vivo inhibition of myeloproliferative neoplasm (MPN) or AML stem cell propagation; or
the in vivo inhibition pre-leukemia stem cell (pre-LSC) transformation into leukemia stem cells (LSCs),
the in vivo inhibition of splicesomes in AML or MPN, and/or
the in vivo inhibition transcript binding to a component of a spliceosome binding pocket or or binding to ADAR1 (adenosine deaminase acting on RNA-1).

In alternative embodiments, provided are pharmaceutical compositions, formulations or therapeutic combinations, comprising 17S-FD-895 (or rebecsinib), or 17S-FD-895 and at least one second drug, for use in the manufacture of a medicament or a pharmaceutical composition for:
treating and ameliorating a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM);
in vivo inhibition of myeloproliferative neoplasm (MPN) or AML stem cell propagation; or
the in vivo inhibition pre-leukemia stem cell (pre-LSC) transformation into leukemia stem cells (LSCs),
the in vivo inhibition of splicesomes in AML or MPN, and/or
the in vivo inhibition transcript binding to a component of a spliceosome binding pocket or binding to ADAR1 (adenosine deaminase acting on RNA-1).

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

FIG. 1A-F illustrate that rebecsinib (17S-FD-895) significantly inhibited human CD34+Lin− (hCD34⁺) cell engraftment in secondary AML patient 50261 (sAML50261) transplanted NSG-SGM3 mouse models that secrete human stem cell factor, GM-CSF and IL-3:

FIG. 1A graphically illustrates the percentage of engrafted human CD45+ (hCD45+) cells in the peripheral blood of immunocompromised mice transplanted with secondary AML patient 37 (sAML37PB) after treatment with vehicle control, fedratinib, rebecsinib (17S-FD-895) or a combination of fedratinib and rebecsinib (17S-FD-895);

FIG. 1B graphically illustrates % CD45 positive (human CD45+ (hCD45+) cells in the spleens of sAML transplanted mice (sAML37SP) mice after treatment with vehicle control, fedratinib, rebecsinib (17S-FD-895) or a combination of fedratinib and rebecsinib (17S-FD-895); and

FIG. 1C graphically illustrates % human CD45 positive cells (hCD45+) in the bone marrow of secondary AML patient 37 transplanted mice (sAML37BM) after treatment with vehicle control, fedratinib, rebecsinib (17S-FD-895) or a combination of fedratinib and rebecsinib (17S-FD-895);

FIG. 1D graphically illustrates % CD34 positive, Lin negative, cells (CD34+ (hCD34⁺ Lin⁻) cells in mice transplanted with secondary AML patient 37 peripheral blood (sAML37PB) after treatment with vehicle control, fedratinib, rebecsinib (17S-FD-895) or a combination of fedratinib and rebecsinib (17S-FD-895);

FIG. 1E graphically illustrates % CD34 positive Lineage negative cells (hCD34⁺Lin⁻ cells) in the spleens of secondary AML patient 37 (AML37SP) transplanted mice after treatment with vehicle control, fedratinib, rebecsinib (17S-FD-895) or a combination of fedratinib and rebecsinib (17S-FD-895); and,

FIG. 1F graphically illustrates % CD34 positive Lineage negative cells (hCD34+) in bone marrow of secondary AML patient 37 transplanted mice (sAML37BM) after exposure to vehicle control, fedratinib, rebecsinib (17S-FD-895) or a combination of fedratinib and rebecsinib (17S-FD-895);

where all p values were calculated using an unpaired T test.

FIG. 2 graphically illustrates that spleen size in combination treatment groups is significantly smaller following primary transplantation, showing spleen weight in mg in secondary AML patient 37 transplanted (sAML37) NSG-SGM3 mice that secrete human stem cell factor, GM-CSF and IL-3 after treatment with vehicle control, fedratinib, rebecsinib (17S-FD-895) or a combination of fedratinib and rebecsinib (17S-FD-895); where all p values were calculated using an unpaired T test.

FIG. 3 graphically illustrates a reduction in serially transplantable leukemia stem cells (LSC) as indicated by a significant decrease in the spleen size of RAG2-/-gc-/-mice that were engrafted with human cells from mice that had been treated with a combination of fedratinib and rebcsinib compared to groups treated with control, fedratinib or rebecsinib (17S-FD-895). For this experiment, live human CD45+ cells from secondary AML patient number 50261 (sAML 50261) engrafted mice were serially transplanted into RAG2-/-gc-/-mice that lack T, B and NK cells following treatment with vehicle control, fedratinib, rebecsinib (17S-FD-895) or a combination of fedratinib and rebecsinib (17S-FD-895); where all p values were calculated using an unpaired T test.

FIG. 4, FIG. 5 and FIG. 6A-C illustrate targeted inhibition of leukemia stem cells with rebecsinib (17S-FD-895), where: rebecsinib inhibits leukemia stem cells (LSC) and reduces AML burden at doses that spare normal hematopoietic stem cells (HSC) in vivo; LSC are exquisitely sensitive to splicing modulation with Rebecsinib in vivo; and, splice isoform biomarkers include SF3B1, MCL1, CD44 and ADAR1:

FIG. 4 graphically illustrates normal human cord blood hematopoietic stem cell (HSC) engraftment in RAG2-/-gc-/-mice based on flow cytometric quantification of the frequency of live CD45 positive cells after administering: vehicle, 10 mg/kg rebecsinib, or 20 mg/kg rebecsinib;

FIG. 5 graphically illustrates leukmia stem cell (LSC) survival based on the frequency of live CD34 positive, CD38 positive, Lin negative progenitor cells (% baseline) after administering: vehicle or rebecsinib; where p value was calculated using an unpaired T test;

FIG. 6A graphically illustrates SF3B1 splicing factor intronic retention by showing SF3B1 intron 2/total expression ratio after administering: vehicle or rebecsinib; where p value was calculated using an unpaired T test;

FIG. 6B graphically illustrates MCL1 levels by showing expression relative to a housekeeping gene, HPRT, after administering: vehicle or rebecsinib; where p value was calculated using an unpaired T test; and

FIG. 6C graphically illustrates CD44-012 levels by showing expression relative to HPRT, after administering: vehicle or rebecsinib; where p value was calculated using an unpaired T test.

FIG. 7, FIG. 8 and FIG. 9 illustrate that the lentiviral MAPT-GFP/RFP splicing reporter reveals intron retention following rebecsinib treatment, and flow cytometry shows decreased ADAR p150 splice isoform expression following rebecsinib treatment:

FIG. 7 illustrates an image ADAR1 p150 positive live cells, inset shows 100 μm measure;

FIG. 8 graphically illustrates the ratio of RFP (MFI) to GFP (MFI) after administering DMSO vehicle control and 100 nM rebecsinib; where p value was calculated using an unpaired T test; and

FIG. 9 graphically illustrates an ADAR1 P150 flow cytometry of an absolute count of ADAR1 P150 positive live cells after administering DMSO vehicle control and 100 nM rebecsinib; where p value was calculated using an unpaired T test.

FIG. 10, FIG. 11, FIG. 12, FIG. 13, FIG. 14, FIG. 15 and FIG. 16 illustrate that inflammatory cytokine signaling drives ADAR1 p150 protein induced splicing alterations, and rebecsinib (17S-FD-895) inhibits high-risk myelofibrosis hematopoietic progenitor cell (HPC) survival at doses that inhibit ADAR1 activity by ADAR1 nanoluciferase reporter assay:

FIG. 10 schematically illustrates the effect of APOBEC3C on pre-LSC cells, and the effect of ADAR p150 on LSC cells;

FIG. 11 graphically illustrates rebecsinib inhibits ADAR1-p150 in a dose dependent manner as shown by qRT-PCR, showing relative mRNA expression normalized to human HPRT after administering none (control), or varying levels of rebecsinib;

FIG. 12 graphically illustrates that rebecsinib inhibits AML patient number 193 (AML 193) progenitor ADAR1p150 protein levels in a dose dependent manner as shown by flow cytometry, showing amounts of ADAR1p150 mean fluorescent intensity (MFI) live cells normalized to DMSO control after exposure to varying amounts of rebecsinib;

FIG. 13 graphically illustrates that rebecsinib inhibits ADAR1 nano-luciferase reporter activity at 1 microM in high-risk myelofibrosis progenitors, showing, using a ADAR1 nano-luciferase reporter, relative luciferase units normalized to cell viability after administering DMSO control, or 1 μM rebecsinib;

FIG. 14 graphically illustrates that rebecsinib inhibits myelofibrosis progenitor clonogenic (HPC) capacity showing CD34 positive, CD38 positive, cells after administering DMSO control, or 1 μM rebecsinib;

FIG. 15 graphically illustrates that rebcsinib inhibits ADAR1 reporter activity in HPC progenitors at 1 microM rebecsinib levels, showing ADAR-1 p150 MFI live Lin negative, and; and CD34 positive, CD38 positive, cells after administering DMSO control, or 1 μM rebecsinib; and,

FIG. 16 graphically illustrates that rebcsinib alters splicing reporter activity in high risk myelofibrosis (hrMF) in samples with high ADAR1 expression, showing RFP MFI live lineage CD34 positive, CD38 positive, cells after administering DMSO control, or 1 μM rebecsinib.

FIG. 17 illustrates an IVIS imaging of ADAR1 Nano-luciferase-GFP (ADAR1-nano-luc) reporter activity marks normal aged human hematopoietic stem cell engraftment in RAG2-/-gc-/-mice transplanted intrahepatically at birth and imaged at 22 weeks post-transplantation.

FIG. 18 illustrates an IVIS imaging of ADAR1 nano-luc reporter stably transduced triple negative breast cancer cells (MDA-MB-231) injected into the cerebral ventricles of newborn RAG2-/-g c-/-mice engraft in the brain and spinal cord.

FIG. 19 illustrates an IVIS imaging at 6 weeks post-transplantation intrahepatically in newborn RAG2−/−g c−/− mice with ADAR1 nano-luc reporter stably transduced triple negative breast cancer cells (100,00 MDA-MB-231 cells per mouse).

FIG. 20 schematically illustrates an exemplary method for making 17S-FD-895 (rebecsinib).

FIG. 21A schematically illustrates that the synthesis of 17S-FD-895 (1) arises through the coupling of side chain 2 and core 3; and that the 11 sp3 stereocenters and stereochemistry of the three olefins of 1 arose from 12 precursors (inset) that are available on the kilogram scale.

FIG. 21B schematically illustrates a retro-analysis of the related macrolide, pladienolide B; shaded/colored highlights denote the sourced components as shown in gray (middle) inset.

FIG. 22A-E, FIG. 23A-F and FIG. 24A-E, graphically illustrate flow cytometric analysis plots of ADAR1-nano-luc-GFP reporter expression (Y-axis) and CD44-APC (a breast cancer stem cell marker) on the X-axis in:

FIG. 22A-C: liver and spinal cord cells,

FIG. 23A-F: peripheral blood (PB), spleen (SP) and bone marrow (BM), and

FIG. 24A-E spinal cord and brain,

showing engraftment in the spinal cord but not the brain following intrahepatic as opposed to cerebral ventricle transplantation.

FIG. 25A-B graphically illustrate stromal co-culture assays in sAML (splicing factor mutated versus unmutated) and high-risk myelofibrosis (MF) versus aged-matched normal bone marrow (a-NBM) samples treated with 17S-FD-895:

FIG. 25A graphically illustrates results showing a significant reduction in sAML LSC (n=B unique samples, including splicing factor mutated and unmutated samples) survival (upper panel) and self-renewal (lower panel) after treatment with rebecsinib compared with a-ICBM (n=4); and

FIG. 25B graphically illustrates results of an a-NBM versus (vs) sAML vs sAML non-mutated, showing dose response of ABM versus (vs) MR vs sAML, results showing a significant reduction in high-risk MF pre-malignant progenitor survival (upper panel) and self-renewal (lower panel) after treatment with rebecsinib compared with a-NBM (n=4). Grouped sAML LSC (n=9) are shown for reference,

statistical analyses were performed using one-way ANOVA.

FIG. 26A-B graphically illustrate in vitro stromal co-culture and protein expression assays in lentiviral ADAR1 reporter-transduced myelofibrosis (MF) samples treated with rebecsinib, where intracellular flow cytometry-based quantification of ADAR1p150 protein expression (FIG. 26A) and STAT3 phosphorylation (FIG. 26B) is shown, as expressed by mean fluorescence intensity, MF/, values within HPC populations; Statistical analyses were performed using two-sided pair-wise t-test; Error bars represent means+/−SEM.

FIG. 27A-D graphically illustrate ADAR1 expression and activity, and pro-survival transcript expression, in AML cells treated with Rebecsinib in vitro:

FIG. 27A graphically illustrates total ADAR1. mRNA expression in lentiviral-shCtrl or shADAR1-transduced AML-193 cells treated with 17S-FD-895 or DMSO control;

FIG. 27B graphically illustrates RNA editing activity on the endogenous LSC-associated transcript AZIN1. quantified by RNA-editing site-specific-qPCR (RESSqPCR) in AML-193 cells treated with 17S-FD-895 or DMSO control for 4 (left panel) or 24 (right panel) hours (hrs);

FIG. 27C graphically illustrates splice isoform-specific qPCR showing reduced MCL1-L expression after ADAR1 knockdown and/or 17S treatment for 4 (left panel) or 24 (right panel) hrs; and

FIG. 27D graphically illustrates splice isoform-specific qPCR showing reduced CD44v3 expression after ADAR1 knockdown and/or 17S treatment for 4 hrs; where for FIG. 27A-D:

statistical analyses were performed using unpaired, two-tailed Student's t-test, n=4 replicates per condition.

FIG. 28A-E graphically illustrate 17S-FD-895 treatment in lentiviral splicing reporter assays, where KG1a or MOLM13 human adult leukemia cells were stably transduced with the pCDH-EF1a-lRES-Puro MAPT splicing reporter lentiviral vector:

FIG. 28A-B graphically illustrate intron retention (FIG. 28A) and viability of KG-1a (FIG. 28B) cells treated with 17S-FD-895 for 24 hrs;

FIG. 28C-E graphically illustrate data from studies where MOLM13-MAPT cells were engrafted into irradiated NSG-SGM mice, followed by 24 hr treatment with vehicle (n=3) and 17S-FD-895 (10 or 20 mg/kg; n=2, n=3), the ratio of RFPIGFP median fluorescence intensity (MF/) in live, CD45+ cells in bone marrow (FIG. 28C), spleen (FIG. 28D), and peripheral blood (FIG. 28E) was quantified by flow cytometry, where p<0.05 by two-tailed Student's t-test.

Like reference symbols in the various drawings indicate like elements.

### DETAILED DESCRIPTION

In alternative embodiments, provided are methods for treating and ameliorating a cancer, for example, a leukemia such as acute myeloid leukemia (AML), comprising administration to an individual in need thereof a pharmaceutical composition comprising 17S-FD-895 (also called rebecsinib), and optionally a second drug such as an ATP-competitive protein tyrosine kinase inhibitor such as fedratinib or dasatinib. In alternative embodiments, provided are methods for the in vivo inhibition of myeloproliferative neoplasm (MPN) or AML stem cell propagation comprising administration to an individual in need thereof a pharmaceutical composition comprising 17S-FD-895 (rebecsinib), or 17S-FD-895 and second drug. In alternative embodiments, provided are methods for the in vivo inhibition pre-leukemia stem cell (pre-LSC) transformation into leukemia stem cells (LSCs) comprising administration to an individual in need thereof a pharmaceutical composition comprising 17S-FD-895 (rebecsinib), or 17S-FD-895 and second drug.

We have demonstrated that 17S-FD-895 (rebecsinib) significantly reduces the frequency of self-renewing CSCs in MPN, MDS and both adult and pediatric AML stromal co-cultures as well as in humanized AML mouse models at doses that spare normal hematopoietic stem cell (HSC; CD34+Lin−) survival and differentiation into mature progeny. Functionally, 17S-FD-895 and related compounds, reduce malignant regenerating potential in vitro and in vivo by impairing CSC self-renewal capacity while sparing normal human hematopoietic stem and progenitor cells (HSPCs). Thus, 17S-FD-895 exhibits a favorable therapeutic index and has potential utility to prevent disease relapse in human hematologic as well as other malignancies.

Splicing modulators, such as E7107, H3B-8800 and 17S-FD-895 (rebecsinib), have efficacy in pre-clinical models of leukemia but only 17S-FD-895 is stable, safe and well tolerated in pre-clinical models. While one chemically distinct splicing modulator, E7107, entered clinical trials for solid tumors (Hong 2014), two of 26 patients developed reversible optic neuritis (Leon 2017) related to compound instability and resulting in early trial discontinuation. Subsequent analysis suggested that ocular toxicity was related to metabolic decomposition of E7107 to a seco-acid. In contrast, the candidate 17S-FD-895 undergoes metabolic decomposition, but without toxic consequences. 17S-FD-895 offers the potential to evaluate splicing modulators in a clinical setting without liabilities associated with compound instability and unoptimized pharmacology. In addition, E7107 showed limited efficacy in clinical trial results (Hong, 2014) whereas there is a significant reduction in AML CSC survival and self-renewal after treatment with 17S-FD-895 in preclinical models.

At a molecular level, 17S-FD-895 modulates splicing of several BCL2 pro-survival family members, including BCL2, MCL1, and BCL-XL (BCL2L1). This provides a novel avenue through which to modulate this class of molecules, some of which are targets of other clinical therapeutics (e.g., venetoclax). BCL2L1 splice isoform switching contributes to LSC generation (Goff et al., 2013) and is of particular relevance to the unique mechanism of action (MOA) of 17S-FD-895 in sAML because its transcripts have been shown to be resistant to another splicing modulatory agent that previously entered clinical trials, E7107 (Aird et al., Nature Communications 2019). Moreover, we previously showed that sAML CSC upregulate the splicing regulatory gene ACIN1 (Crews et al., 2016), which regulates production of BCL2L1 (BCL-XL) in the exon junction complex (EJC) (Michelle et al., 2012). Moreover, MCL1L is overexpressed in high risk MPNs and MDS, sAML, pediatric AML, and multiple myeloma. In addition, IL-6ST, ADAR1p150 and STAT3beta splice isoforms are overexpressed during MDS and MPN progression to AML. Thus, hematologic malignancies may be particularly sensitive to splicing modulator treatment with 17S-FD-895 due to intrinsic activation of these pro-survival and self-renewal promoting isoforms in CSC, and subsequent modulation by 17S-FD-895. Quantification of CSC-specific and splicing modulator-responsive transcripts will facilitate rapid screening of in vivo sensitivity of different tissues to splicing modulation and identification of doses that exhibit minimal effects on normal tissues compared with malignant cell types.

A Phase 1 trial in patients with MDS, AML and CMML was conducted with another chemically distinct splicing modulator, H3B-8800. H3B-8800 is a splicing factor mutation-dependent splicing modulatory agent. The most common mutations observed were in RNA splicing factors SF3B1, U2AF1, SRSF2 (88% of patients). In addition, there was no CR or PR observed in the trial showing a lack of efficacy with this splicing modulator. In contrast, through extensive medicinal chemical and toxicology studies, we have identified 17S-FD-895 (Kumar 2016) as a stable, well-tolerated FD-895 analog that is spliceosome mutation independent.

The clinical feasibility with 17S-FD-895 (rebecsinib) is based on extensive in vitro and in vivo pre-clinical studies showing stability, safety, tolerability and human CSC-targeting efficacy in humanized models of AML. Specifically, 17S-FD-895 has a favorable safety profile with no ocular toxicity, as determined by a board certified ophthalmologist, observed in rats, rabbits and monkey toxicity studies. 17S-FD-895 has a favorable potency and therapeutic index compared to other splicing modulators in that it reduces disease regenerating potential in in vitro and in vivo by impairing CSC self-renewal capacity while sparing normal human HSPC. Finally, 17S-FD-895 has a clinically tractable formulation and pharmacological (PK/PD) properties with favorable bioavailability and stability that enables twice weekly intravenous dosing regimens that eliminate human CSCs. Sensitive and selective methods to predict and monitor response to splicing modulator therapy with 17S-FD-895 will be essential to the future clinical development of these agents.

While the invention is not limited by any particular mechanism of action, 17S-FD-895 (rebecsinib) inhibits transcript binding to SF3B1 and other components of the spliceosome binding pocket, including SF3B3 and PHF5A, which perturbs generation of key CSC survival transcripts, thereby inducing effective elimination of CSCs in AML samples regardless of splicing factor mutational status. This may broaden the spectrum of activity against other malignancies with splicing deregulation and contrasts with another splicing modulatory agent, H3B-8800, which is dependent on splicing factor mutations, such as SRSF2, for altering its RNA binding preference within the spliceosome and efficacy (https://ashpublications.org/blood/article/134/Supplement_1/673/426543/Results-of-a-Clinical-Trial-of-H3B-8800-a-Splicing).

Splice isoform-specific molecular analyses characterizing the effects of 17S-FD-895 in AML models demonstrate that mechanistically, this compound potently disrupts the spliceosome, inducing quantifiable and reproducible intron retention and exon skipping events detectable in disease-relevant (MCL1, CD44) and spliceosome-associated biomarkers (SF3B family). Functionally, we tested the relative in vitro sensitivity of primary patient-derived sAML CSC to 17S-FD-895 compared with normal age-matched hematopoietic stem and progenitor cells (HSPC) and determine the effects of splicing modulation on sAML CSC survival and self-renewal capacity. We also tested the in vitro effects of 17S-FD-895 on the hematopoietic differentiation potential of normal human HSPC by comparing the in vitro effects of 17S-FD-895 on normal immune cell survival and development.

Biomarkers were developed and validated to monitor responses to splicing modulation in human and rat cells. The transcripts selected for species-specific primer design included MCL1, BCL-XL, CD44, PTK2B, DNAJB1, and the SF3B family. Together, these biomarkers are diagnostic tools to predict and monitor response to splicing modulator treatment.

### Making 17S-FD-895

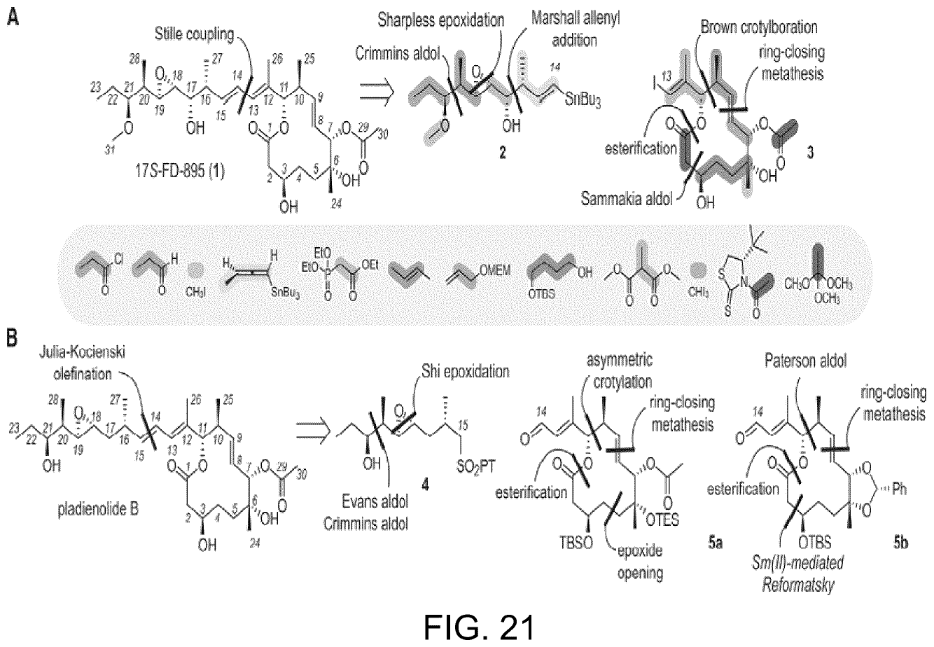

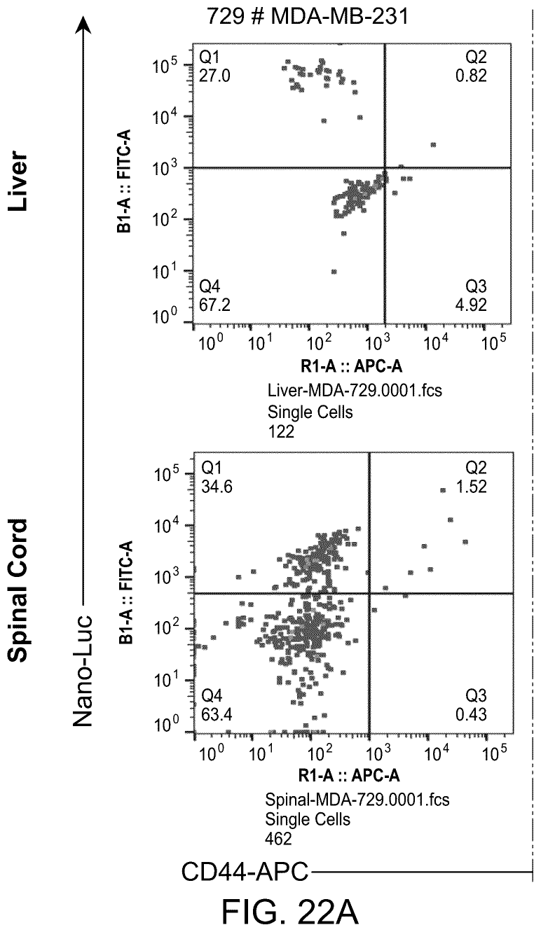

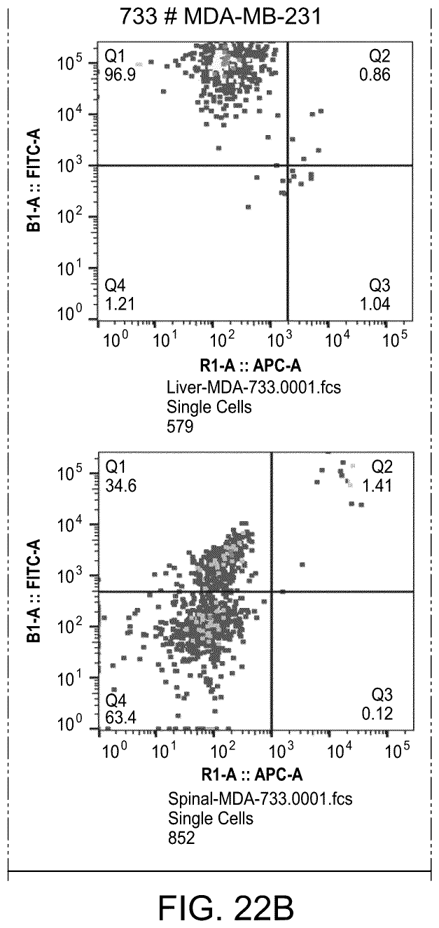

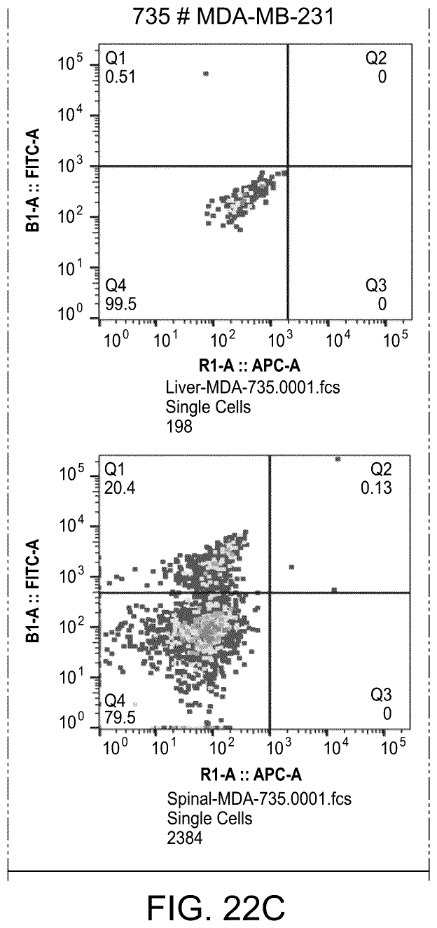

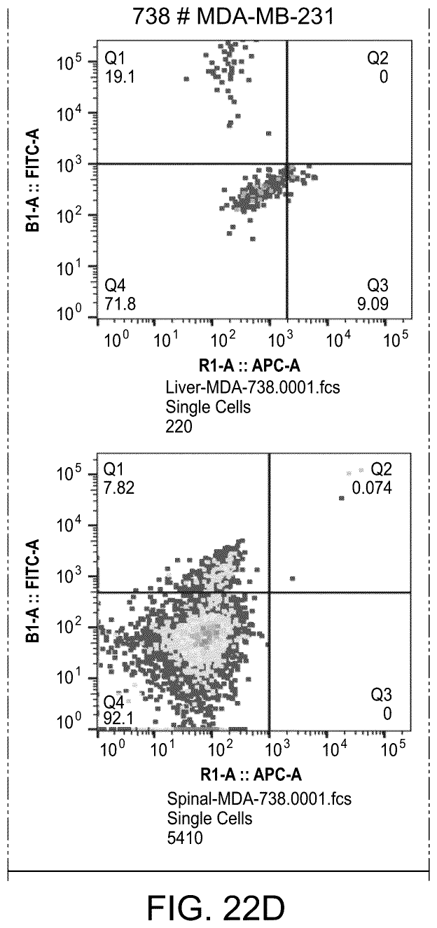

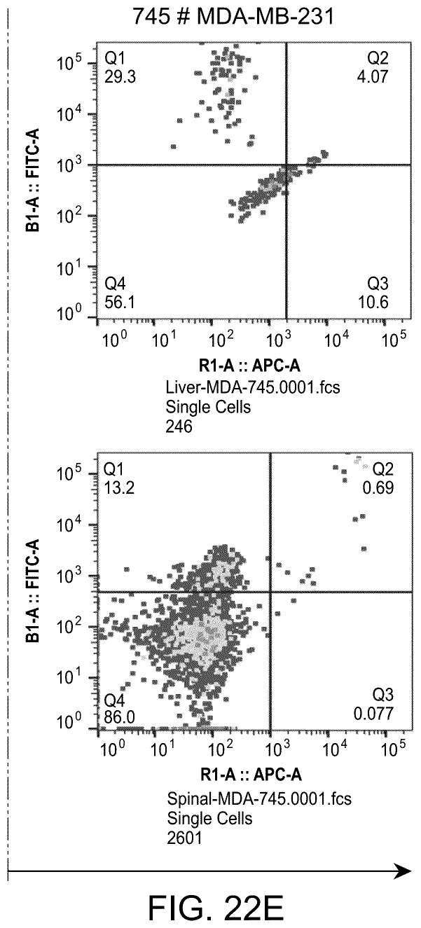

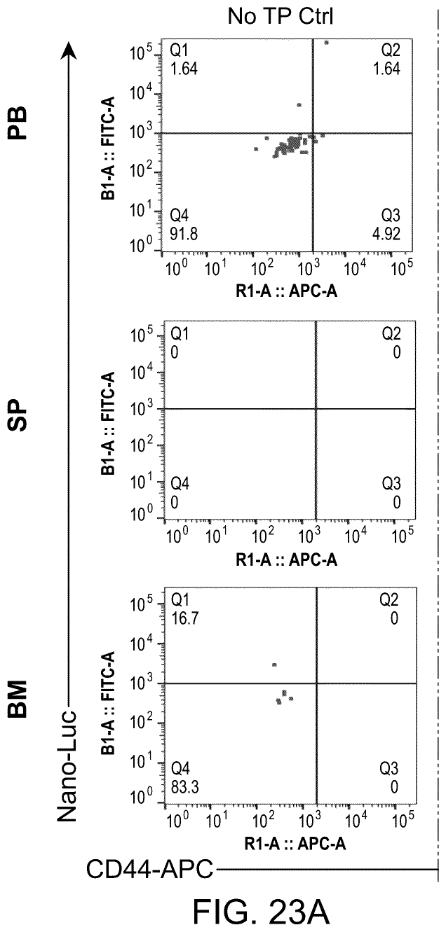

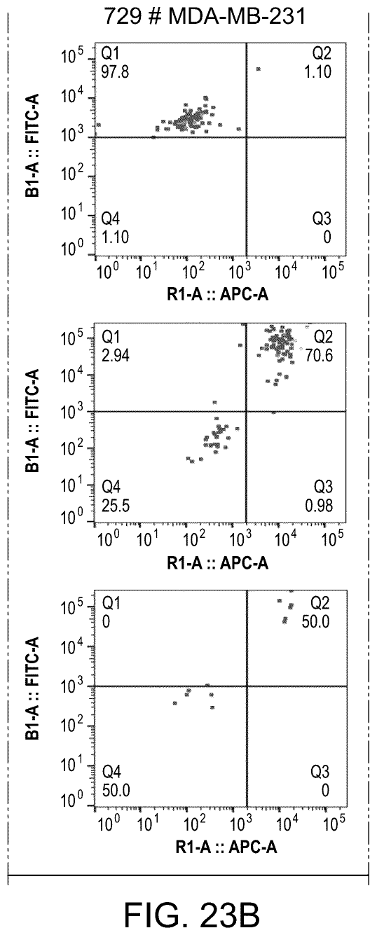

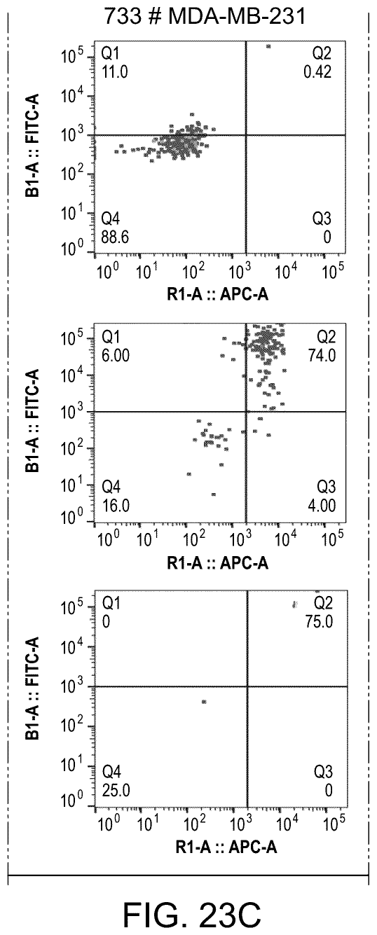

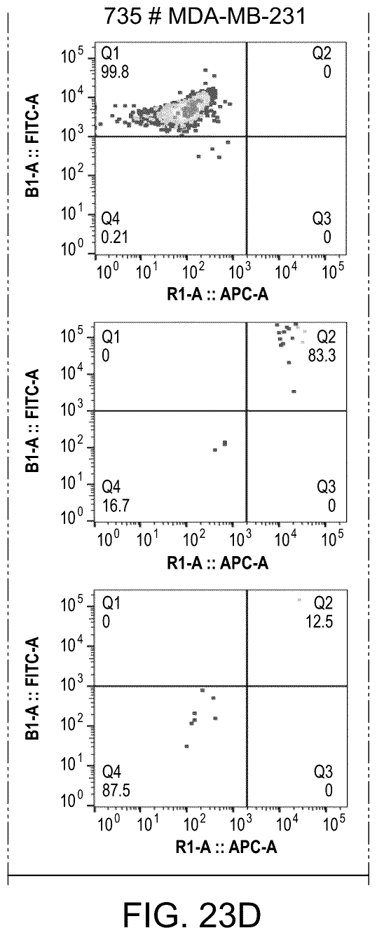

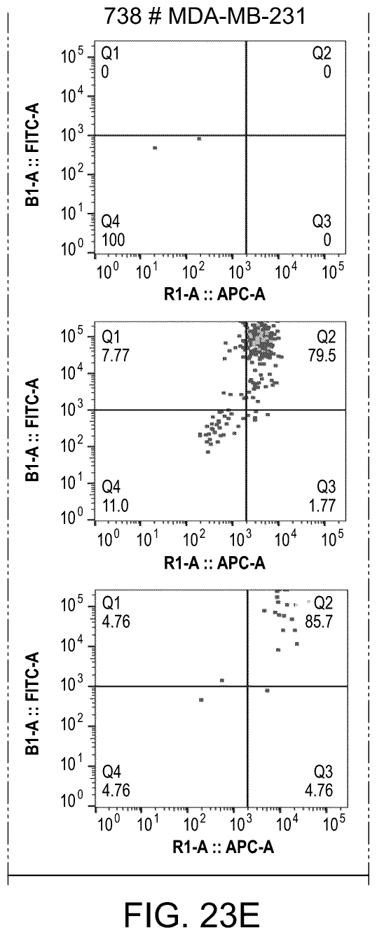

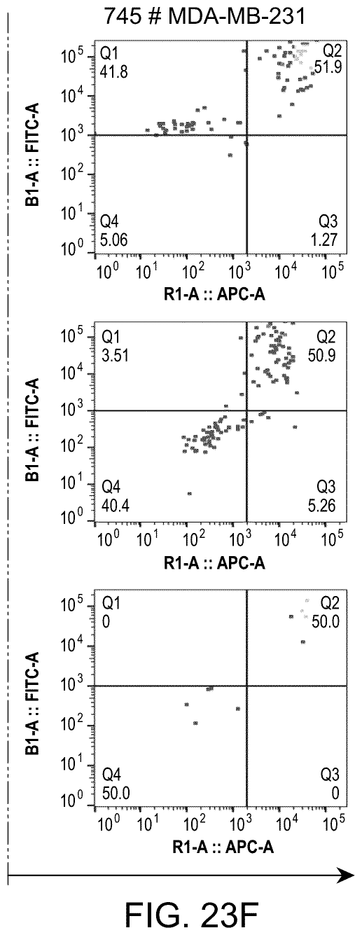

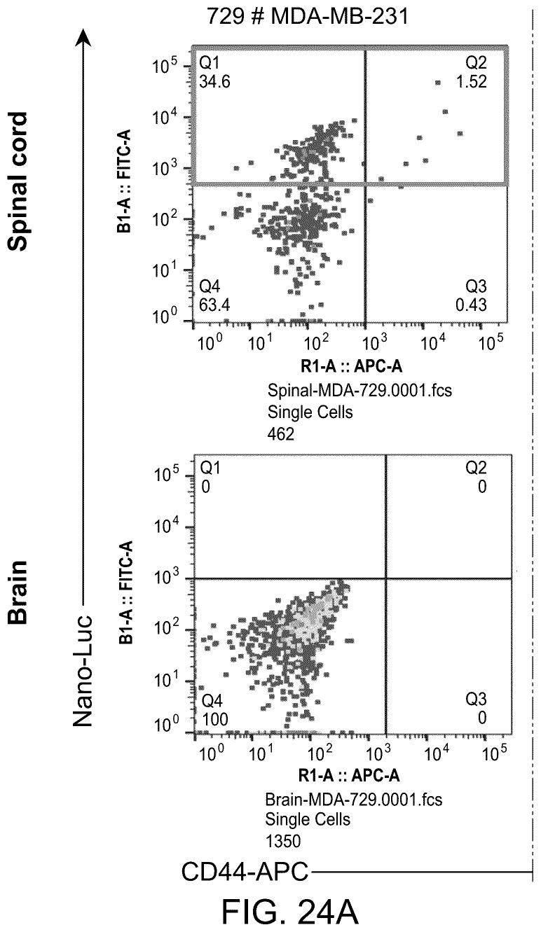

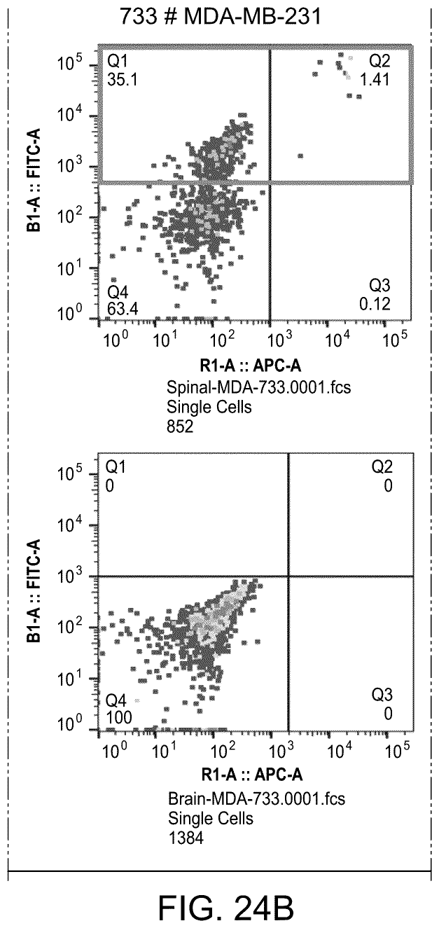

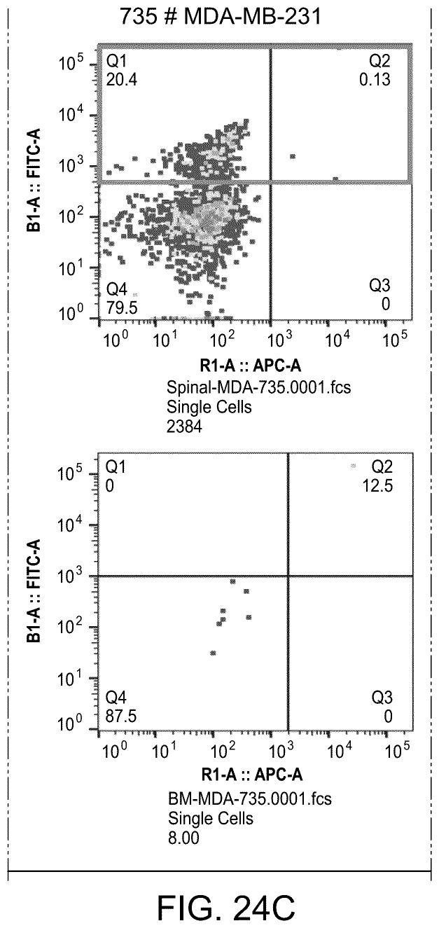

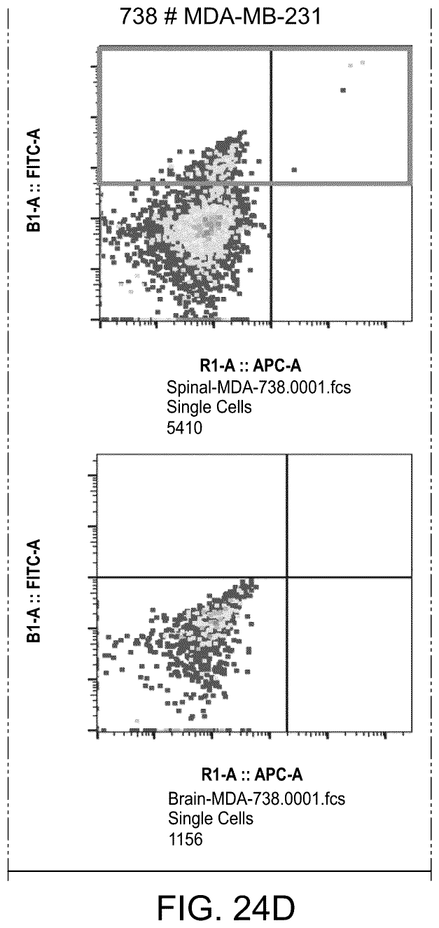

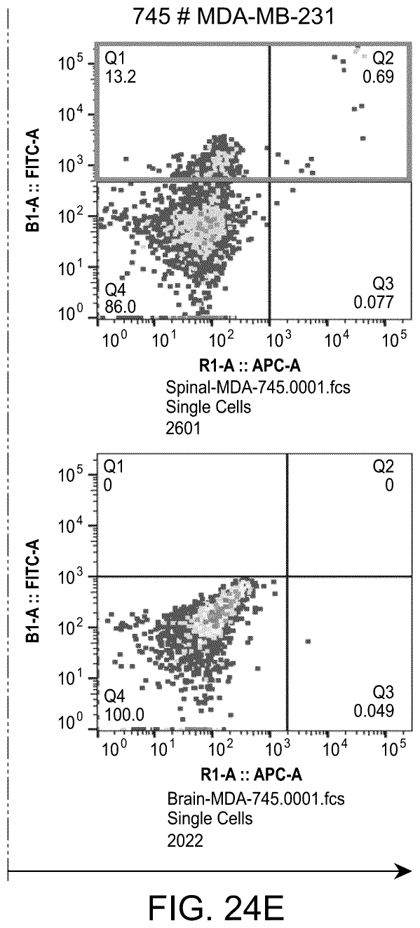

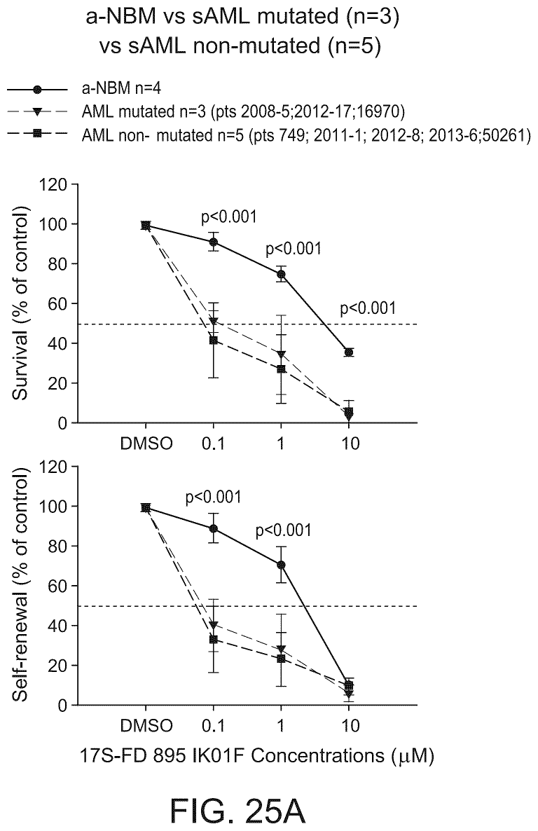

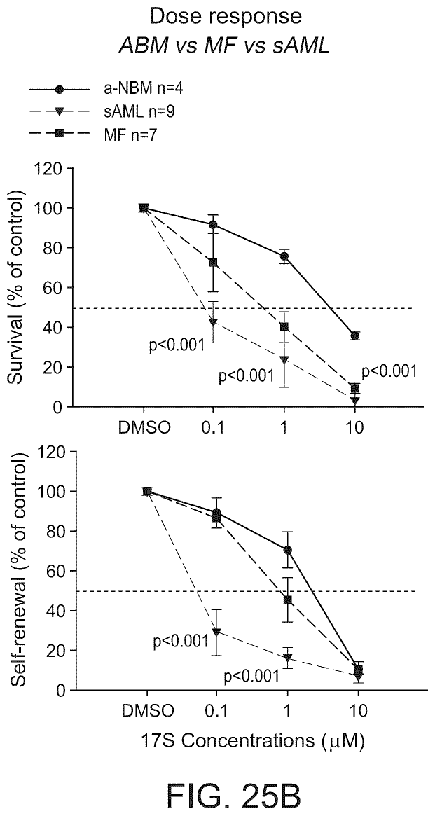

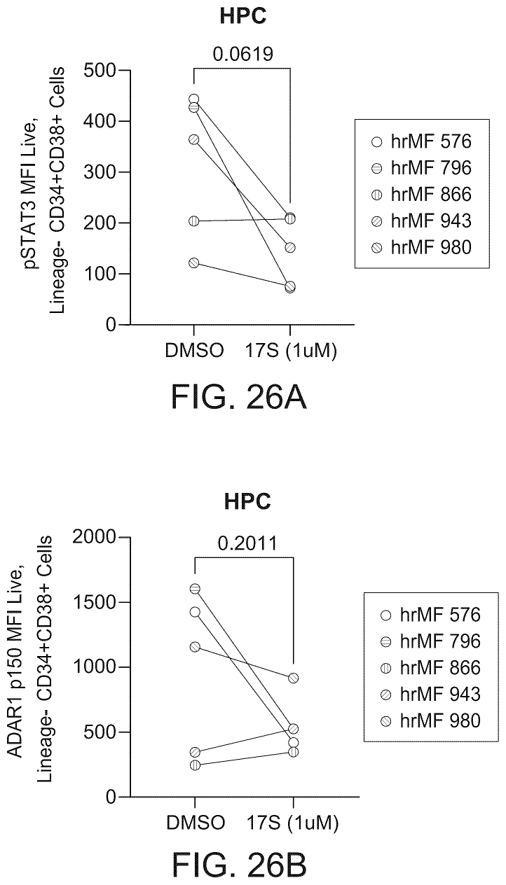

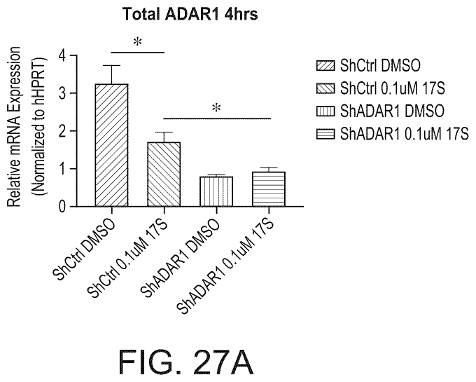

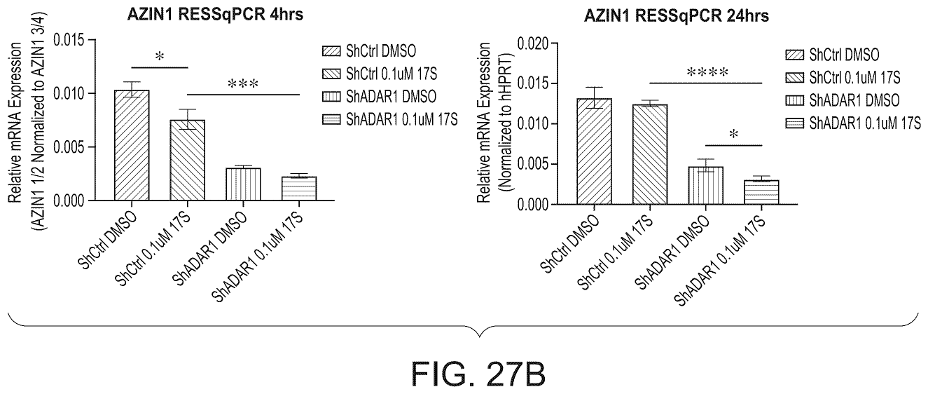

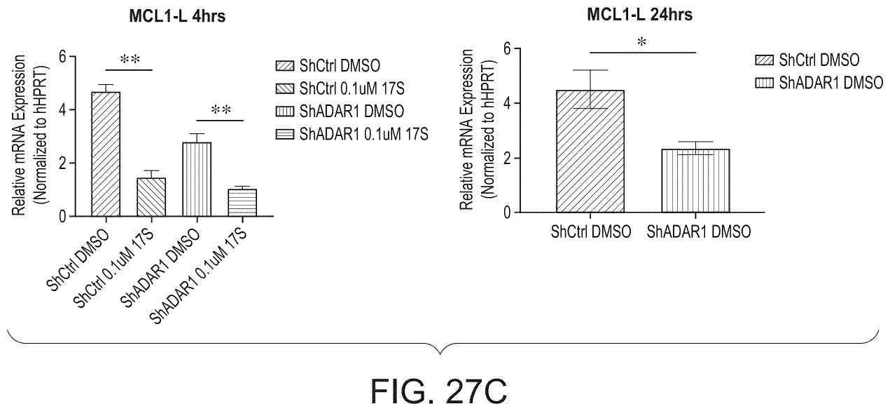

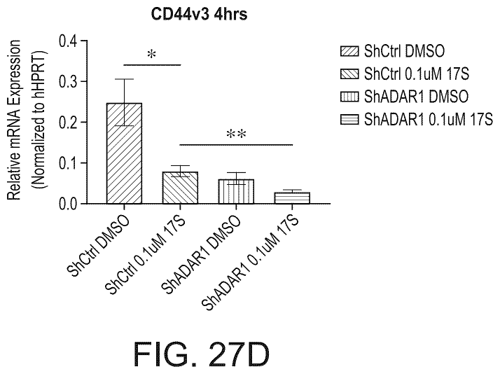

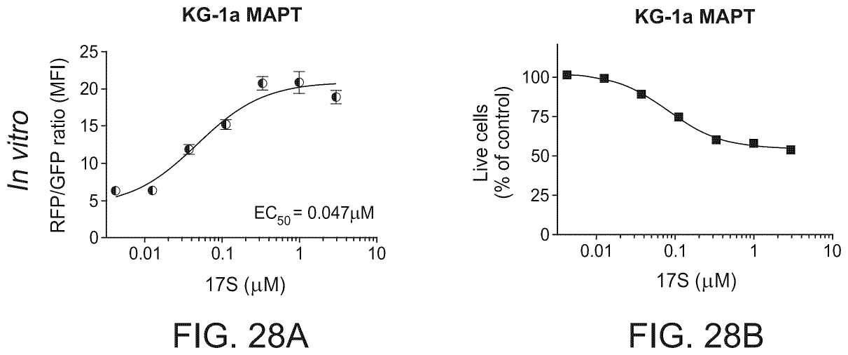

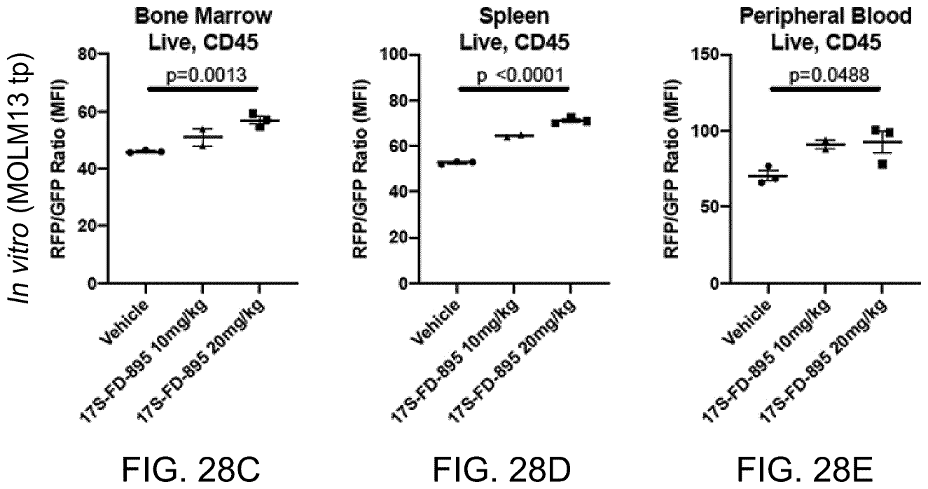

17S-FD-895 (rebecsinib) can be made using any protocol known in the art, for example, as described in Chan et al., Cell Reports Physical Science 1: see FIG. 21, and as described in FIG. 22A-B:
FIG. 21A schematically illustrates that the synthesis of 17S-FD-895 (1) arises through the coupling of side chain 2 and core 3. The 11 sp3 stereocenters and stereochemistry of the three olefins of 1 arose from 12 precursors (inset) that are available on the kilogram scale. The key steps used to prepare each component are noted.
FIG. 21B schematically illustrates a retro-analysis of the related macrolide, pladienolide B, as developed by Ghosh and Anderson²⁴ from core 5a and Kotake²⁸ from core 5b. Shaded/colored highlights denote the sourced components as shown in gray (middle) inset.

### Formulations and Pharmaceutical Compositions

In alternative embodiments, provided are pharmaceutical formulations or compositions comprising drugs, and therapeutic combinations of drugs, and formulations, and liposomes, for practicing methods and uses as provided herein to treat or ameliorate a cancer, for example, to treat or ameliorate a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM); or can be used for in vivo inhibition of myeloproliferative neoplasm (MPN) or AML stem cell propagation; or can be used for the in vivo inhibition pre-leukemia stem cell (pre-LSC) transformation into leukemia stem cells (LSCs), or can be used for the in vivo inhibition of splicesomes in AML or MPN, and/or can be used for the in vivo inhibition of transcript binding to a component of a spliceosome binding pocket or binding to ADAR1 (adenosine deaminase acting on RNA-1).

In alternative embodiments, a formulation or pharmaceutical compositions used to practice methods and uses as provided herein can be administered parenterally, topically, orally or by local administration, such as by aerosol or transdermally, or intravitreal injection. The formulations and pharmaceutical compositions (including therapeutic drug combinations) can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (for example, any active agent as used in methods provided herein) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions used to practice methods as provided herein, for example, to deliver a drug or drugs, for example to treat or ameliorate a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM); or can be used for in vivo inhibition of myeloproliferative neoplasm (MPN) or AML stem cell propagation; or can be used for the in vivo inhibition pre-leukemia stem cell (pre-LSC) transformation into leukemia stem cells (LSCs), or can be used for the in vivo inhibition of splicesomes in AML or MPN, and/or can be used for the in vivo inhibition of transcript binding to a component of a spliceosome binding pocket or binding to ADAR1 (adenosine deaminase acting on RNA-1). In alternative embodiments, these compositions are designed to target specific molecules, including biologic molecules, such as polypeptides, including cell surface polypeptides, for example, for targeting a desired cell type or organ, for example, a nerve cell or the CNS, and the like.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, a drug combination as provided herein, or a ADAR1-encoding nucleic acid, or a ADAR1 polypeptide), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, a drug or drug combination as provided herein) to a desired cell type (for example, a cancer cell), as described for example, in U.S. Pat. Pub. No. 20070110798.

Provided are nanoparticles comprising compounds (for example, a drug or drug combination as provided herein) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121.

### Delivery Cells and Delivery Vehicles

In alternative embodiments, any delivery vehicle can be used to practice the methods or uses as provided herein, for example, to deliver compositions (for example, a drug or drug combination as provided herein) in vivo, to an individual in need thereof. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In alternative embodiments, a drug or drug combination as provided herein is delivered in vivo using methods as provided herein formulated in a lipid formulation or a liposome and injected for example intramuscularly (IM), for example using formulations and methods as described in U.S. patent application no. US 20210046173 A1; wherein optionally the drug or drugs is/are formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Dosaging

The pharmaceutical compositions, drug combinations and formulations used to practice methods and uses as provided herein can be administered for prophylactic and/or therapeutic treatments, for example, to treat or ameliorate a cancer, wherein optionally the cancer is a leukemia, and optionally the cancer is acute myeloid leukemia (AML), myeloproliferative neoplasm (MPN), myelodysplastic syndrome (MDS), acute myeloid leukemia (AML), chronic lymphocytic leukemia (CLL) and/or multiple myeloma (MM); or can be used for in vivo inhibition of myeloproliferative neoplasm (MPN) or AML stem cell propagation; or can be used for the in vivo inhibition pre-leukemia stem cell (pre-LSC) transformation into leukemia stem cells (LSCs), or can be used for the in vivo inhibition of splicesomes in AML or MPN, and/or can be used for the in vivo inhibition of transcript binding to a component of a spliceosome binding pocket or binding to ADAR1 (adenosine deaminase acting on RNA-1); and, the pharmaceutical compositions, drug combinations and formulations used to practice methods and uses as provided herein can be administered to an individual in need thereof in an amount sufficient to treat, ameliorate, protect against, reverse or decrease the severity or duration of the cancer.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The pharmaceutical compositions, drug combinations and formulations used to practice methods and uses as provided herein can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the drug or drugs are contained in a nanoparticle, a particle, a micelle or a liposome or lipoplex, a polymersome, a polyplex or a dendrimer. In alternative embodiments, these dosages are administered once a day, once a week, or any variation thereof as needed to maintain in vivo expression levels of a desired drug, which can be monitored by assessing the therapeutic effect, for example, to treat, ameliorate, protect against, reverse or decrease the severity or duration of a cancer. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations, therapeutic drug combinations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary Methods Using 17S-FD-895 to Reduce Self-Renewing AML CSCs and for Targeted CSC Eradication

This example demonstrates that methods and compositions as provided herein are effective in vivo for reducing the levels or generation of self-renewing AML CSCs and for targeted CSC eradication.

We have demonstrated that 17S-FD-895 (also called rebecsinib) significantly reduces the frequency of self-renewing AML CSCs in stromal co-cultures as well as in humanized AML mouse models at doses that spare normal hematopoietic stem cell (HSC; CD34+CD90+Lin−) survival and differentiation into mature progeny. In AML CSC, these effects are accompanied by on-target splicing modulatory effects, including in MCL1 and splicing factor gene products such as SF3B3, which forms part of the splicing modulator binding pocket.

We have also developed and validated sensitive splice isoform biomarkers to detect SF3B family members that are readily quantifiable with qRT-PCR assays. Responses to splicing modulator treatment can also be monitored in live cells using a lentiviral fluorescence splicing reporter, which is a novel tool compatible with use in clinical samples.

Splice isoform-specific molecular analyses characterizing the effects of 17S-FD-895 in AML models demonstrate that mechanistically, this compound potently disrupts the spliceosome, inducing quantifiable and reproducible intron retention and exon skipping events detectable in disease-relevant (MCL1, CD44) and spliceosome-associated biomarkers (SF3B family). In vitro splicing modulator treatment and species-specific splice biomarker validation in human and rat cell lines.

Test System: In vitro cell line assays under treatment with 17S-FD-895 (1 nM-10 uM ranges). For in vitro treatment, human leukemia cell lines (KG-1a, MOLM-13, and HL-60) and rat leukemia lines (RBL-1) were treated with 17S-FD-895 for 4 hrs in standard cell culture media for each line. Cells were harvested for RNA extraction and processed for qRT-PCR with splice isoform-specific primers designed to recognize intron retention or exon-skipping events in species-specific transcripts of SF3B family members, MCL-1, and other sAML-specific splice variants.
Test Article: 17S-FD-895 (research and pre-pilot batches #IA01f and IK01f).
Control Article: <0.5% DMSO.
Test and Control Article Preparation: Test articles were thawed at room temperature from stock aliquots stored in glass vials at −80 C in 100% DMSO at a concentration of 1 mg/mL. Immediately upon thawing, Test Articles were diluted directly into tissue culture media for final treatment at a concentration of 1 uM for single dose studies, or at a range from 100 nM-10 uM for dose response studies. For vehicle controls, 100% DMSO was diluted directly into tissue culture media at the same dilutions as the Test Article solution.
In-Life Monitoring: Assay measurements included splice isoform-specific, species-specific qRT-PCR.

A selective splicing modulator, 17S-FD-895, and related compounds, exhibit disease-modifying, on-target efficacy against cancer stem cells in diverse human cancer models. We have shown that spliceosome deregulation in cancer stem cells (CSCs) renders them exquisitely vulnerable to the novel splicing modulator, 17S-FD-895 [Crews 2016], which targets the SF3B1 component of the spliceosome [Kotake 2007]. Splicing modulation with 17S-FD-895 significantly reduces secondary acute myeloid leukemia (sAML) CSC burden in vivo with a reduction in sAML CSC survival and self-renewal. Importantly, splicing modulation with 17S-FD-895 spares normal human hematopoietic progenitor cells in vivo and 17S-FD-895 eradicates human CSC, independent of mutation status. Thus, 17S-FD-895 meets a pressing unmet medical need for developing splicing modulator therapy to eradicate CSCs thereby preventing AML therapeutic resistance and relapse.

While this invention is not limited to by particular mechanism of action, mechanistically, 17S-FD-895 binds within the spliceosome adjacent to SF3B1, SF3B3, and PHFSA. Treatment with 17S-FD-895 increases intron retention and exon skipping thereby reducing survival and self-renewal of CSC. In cells from human age-related malignancies including AML and multiple myeloma, these effects are accompanied by on-target splicing modulatory effects, including in pro-survival MCL1L transcripts, and splicing factor gene products such as SF3B1 and SF3B3, which form part of the splicing modulator binding pocket as well as alterations in self-renewal promoting ADAR1 and STAT3beta transcripts.

Here, we describe targeted CSC eradication as well as unique intron-retained and exon-skipped transcripts that can be quantified by splice isoform-specific qRT-PCR, a novel lentiviral splicing reporter assay, and RNA-sequencing analyses and can be used as predictive biomarkers to monitor molecular responses to 17S-FD-895 treatment. A subset of these transcripts can only be detected after exposure to a splicing modulator, and their generation precedes cytotoxic effects in cells, thus enabling quantification of relative sensitivity and predicting response to splicing modulation. Together, these molecular tools provide a sensitive method of detecting activity and mechanism of action of 17S-FD-895, and demonstrate CSC selectivity of 17S-FD-895 in humanized stromal co-cultures and humanized CSC mouse models, which will have utility in future clinical development of this class of therapeutic agents.

In addition to on-target splicing modulatory effects detected in human and rat leukemia cells treated with 17S-FD-895, we also found that human multiple myeloma cells were exquisitely sensitive to spliceosome disruption. SF3B1 and SF3B3 splice biomarkers, along with MCL-1 transcripts, were the most highly expressed and responsive to 17S-FD-895 in human cells.

In in vitro dose response splicing modulator treatment and human-specific splice biomarker analyses in primary sAML patient samples, we then tested the molecular response of CD34+ CSCs isolated from primary sAML patient samples treated in vitro with 17S-FD-895.

We used a lentiviral splicing reporter in human AML CSC for in vitro splicing modulator treatment and detection of intron retention in live cells. For detection of splicing activity in live, primary, human AML CSC, we first transduced human AML CSC (CD34+ cells) with the lentiviral fluorescence splicing reporter. In keeping with results observed in stably-transduced human leukemia cell lines, in human AML CSC cells treated with 17S-FD-895, there was increased intron retention following 17S-FD-895 treatment.

We next determined the effects of splicing modulation in vivo on sAML CSC burden in a clinically-relevant animal model, 2018-PDX50261-IV-2 (unmutated sAML PDX model). We tested an increased frequency of dosing at the previously-used concentration (10 mg/kg) of 17S-FD-895 to evaluate CSC burden after treatment in sAML-engrafted mice. In addition, we determined the effects of splicing modulation on sAML CSC burden in a clinically-relevant animal model, 2019-PDX2008-5-I. Sensitive splice biomarker analyses using primers specific for human transcripts in qRT-PCR assays demonstrated that in vivo 17S-FD-895 treatment triggers intron retention in SF3B family and MCL1 transcripts, and reduces the expression of sAML CSC-specific transcripts. These biomarker studies provide evidence of on-target molecular responses to splicing modulator treatment that can be rapidly quantified and monitored in human cells isolated from leukemia-engrafted animal tissues, suggesting the potential utility of these molecular assays as companion biomarkers in the clinical setting. For human-specific biomarker detection and dose modeling in vivo, we completed splice biomarker studies on cells isolated from sAML PDX50261 mice treated with 17S-FD-895 at doses of 10-20 mg/kg, compared with vehicle control. Splice biomarker analyses in dose escalation studies showed patterns of exon skipping (MCL1-S expression) consistent with on-target splicing modulatory activity of 17S-FD-895, along with increased MCL1-S/L ratios and SF3B family member intron retention, most notably in SF3B3 transcripts. No evidence of Test Article-related toxicity was observed after 2 weeks of dosing at 20 mg/kg in this humanized in vivo model of sAML. Overall, a dose of 10 mg/kg in mice is sufficient to reduce in vivo sAML CSC burden while sparing normal HSPC development. No evidence of Test Article-related toxicity was observed after 2 weeks of dosing at 10 or 20 mg/kg in a humanized in vivo model of normal HSPC development. Our biomarker system confirmed a functional and molecular therapeutic index for 17S-FD-895, whereby sAML CSC are more sensitive to splicing modulation than normal HSPC and their progeny.

To further dissect the role of pre-mRNA splicing of pediatric AML stem and progenitor cells, we performed RNAseq of highly purified non-leukemic or AML hematopoietic stem cells (HSCs; CD34+CD38−Lin−) and hematopoietic progenitor cells (HPCs; CD34+CD38+Lin−) from pediatric bone marrow or peripheral blood. Utilizing a splice variant-specific alignment algorithm we evaluated genome wide alternative splicing events in HSCs and HPCs derived from pediatric patients with AML. We observed differential splicing of all classes of alternative splicing events, including skipping of cassette exons, retained introns and competing 5′ and 3′ splice sites. The pro-survival splice variant, MCL1-L, of the BCL2 family was shown to be highly expressed in pediatric AML progenitors via RNA sequencing and significantly increased via qRT-PCR between CD34+ cord blood and pediatric AML samples. HSCs show both decreased expression levels of RBFOX2 and MBNL1 and MBNL2, while AML derived HPCs suggest antagonistic coregulation of splicing by RBFOX2 and CELF2.

We evaluated the effect of this splicing modulator on pro-survival splice variants in CD34+ cells derived from both peripheral blood as well as bone marrow of pediatric AML patients using qPCR. PCR demonstrated dose-dependent increase in MCL1 exon 2 skipping, producing pro-apoptotic MCL1-S transcripts. Furthermore, splicing modulation induces an increase in SF3B1 intron levels following treatment and reduction of misspliced PTK2B-202. In addition, hematopoietic progenitor assays demonstrated a dose-dependent reduction in clonogenicity and self-renewal in CD34+ cells isolated from pediatric AML samples. Notably, pediatric AML samples were more sensitive to splicing modulation that both de novo AML as well as sAML, whereas normal CB samples were unaffected by splicing modulator treatment.

We conducted further pharmacokinetic, safety and tolerability studies in rats, rabbits and monkeys. In the animal toxicology studies, 17S-FD-895 was well-tolerated at the doses tested. Toxicological testing has included non-GLP single dose tolerability and TK studies in rats, rabbits and monkeys. In rats, dose-dependent changes in hematology parameters were observed which included decreases in mean neutrophil, monocyte, eosinophil, and platelet counts. There was a modest decrease in several hematology parameters including platelets in rabbits. In monkeys, there was a minimal decrease in platelet counts in a dose dependent manner. There were no other significant toxicology findings in the in vivo animal studies.

Previous toxicology work included non-GLP single dose tolerability and TK in rats, rabbits and monkeys. In rats, a minimal, non-statistically significant decrease in monocytes, neutrophils and eosinophils was observed following dosing with 17S-FD-895. There was a minimal decrease in in platelets in female rabbits at doses≥20 mg/kg 17S-FD-895. In monkeys, there no changes in hematological, coagulation or clinical chemistry parameters observed. There were no other significant toxicology findings in the in vivo animal studies. Future work will include non-GLP dose range finding studies in rats and rabbits and GLP definitive tolerability/TK studies in rats and rabbits with clinically relevant route (IV) and schedule (twice weekly for 4 doses). Previous IND-enabling pharmacology studies provided in vivo CSC selectivity data demonstrating that 17S-FD-895 treatment reduces the frequency of CSC in therapy-resistant secondary AML. We have identified sensitive splice biomarkers of in vivo response to splicing modulation with 17S-FD-895 and have shown that treatment with 17S-FD-895 results in significant increases in intron retention (SF3B1), exon skipping (MCL1-S) along with reductions in sAML CSC-specific functional splice biomarkers (CD44-012). Previous studies have also shown that 17S-FD-895 specifically targets CSCs rather than the bulk population in sAML and spares normal hematopoietic stem and progenitor cells and demonstrated that effects of 17S-FD-895 are predicated on spliceosome deregulation independent of splicing factor mutation status.

Finally, PD studies in the PBMCs of monkeys that received 17S-FD-895 showed on-target splicing modulation typified by MCL1 exon skipping and SF3B3 intron retention.

In one study, a patient 72 years old, female, diagnosed with AML, having had no treatment, having:
Mutations: CEBPA, CSF3R, DNMT3A, DNMT3B, EP300, ETV6, EZH2, FANCL, KMT2C, LUC7L2, NOTCH1, NRAS, RPL5, SUZ12, TET2.
Cytogeneics: 46,XX,1,inv(3)(q21q26.2),del(5)(q14q34),der(12)t(1;1 2)(q21;p11.2),20,+r,+mar1 [9]/46,sl,der(7)t(7;9)(p13;q13)[4]/46,sl,i(21)(q10)[3]/46,s1, add(2)(q31)[2]/46,sl,add(2)(q33)[2]

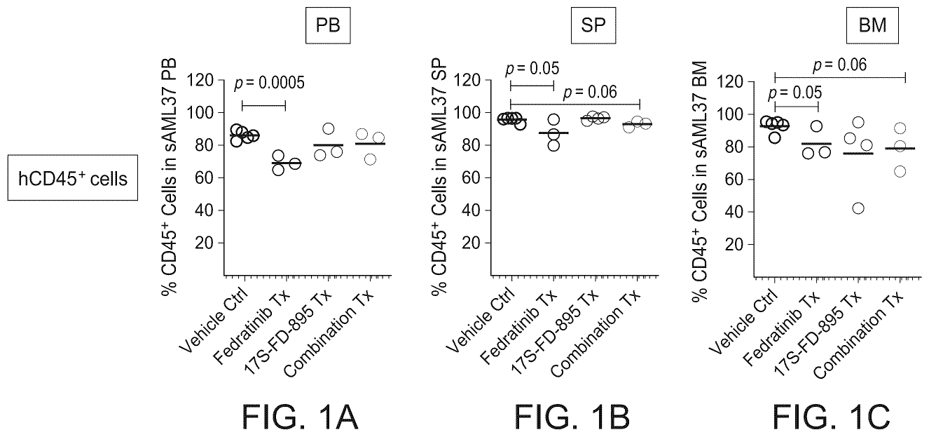

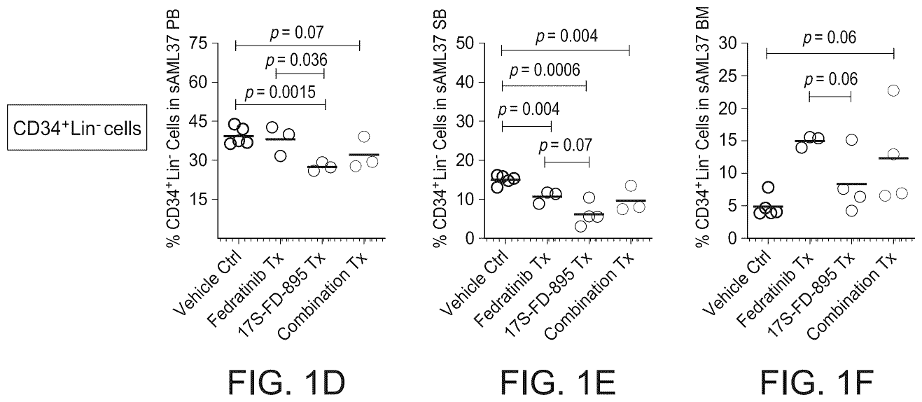

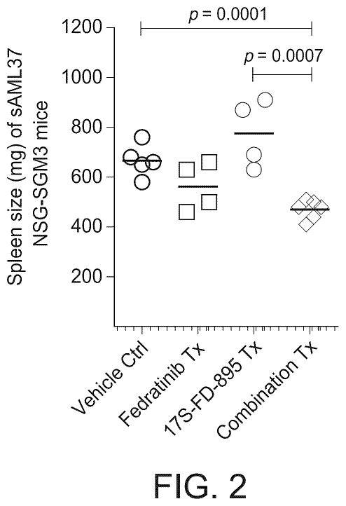

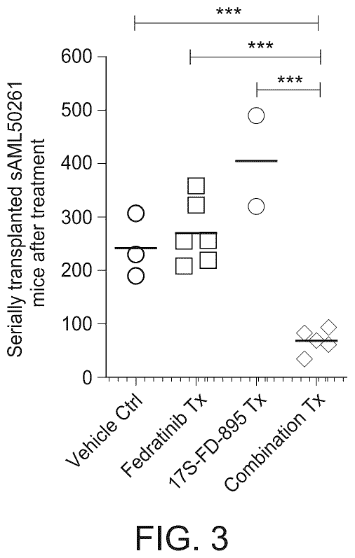

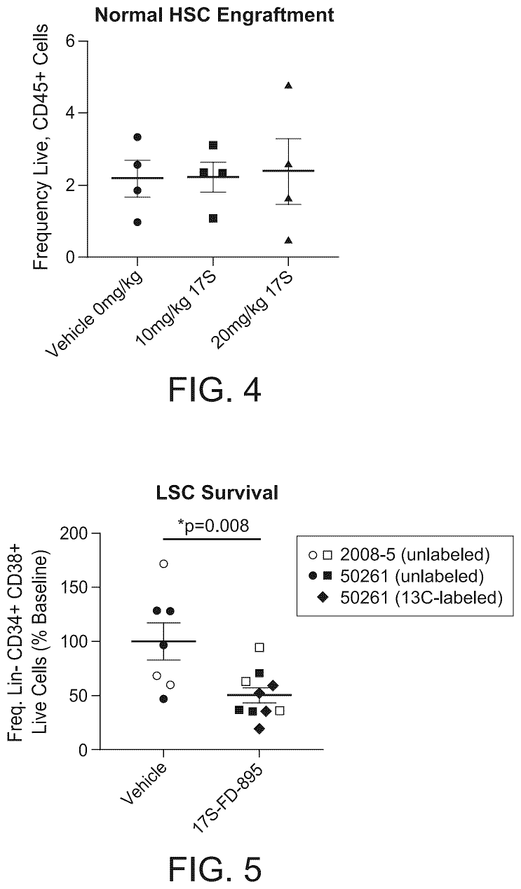

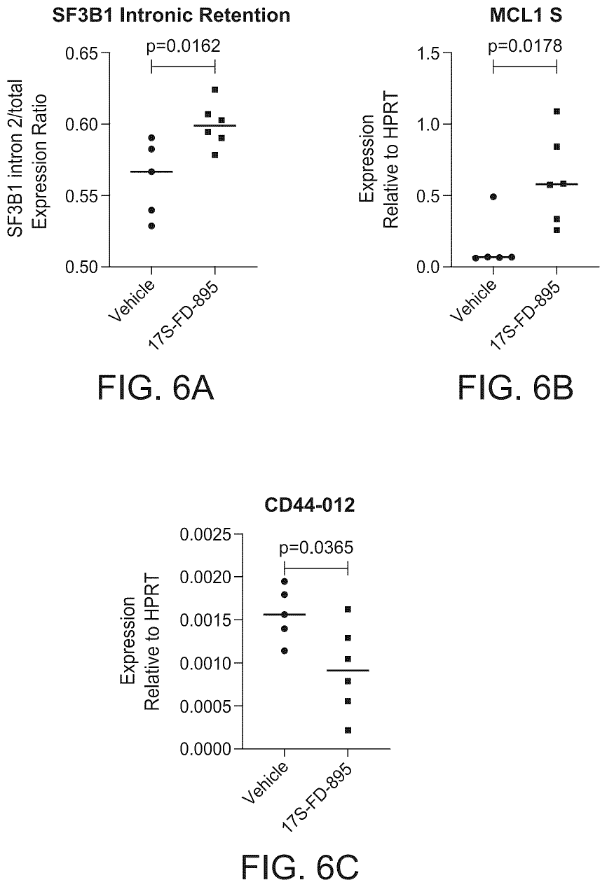

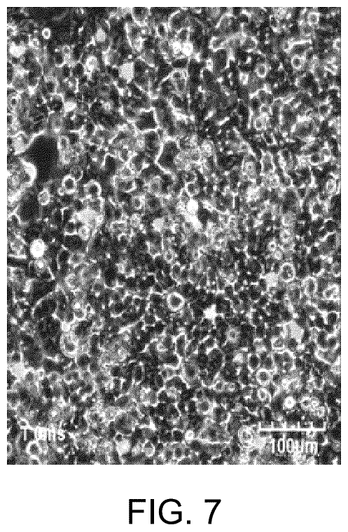

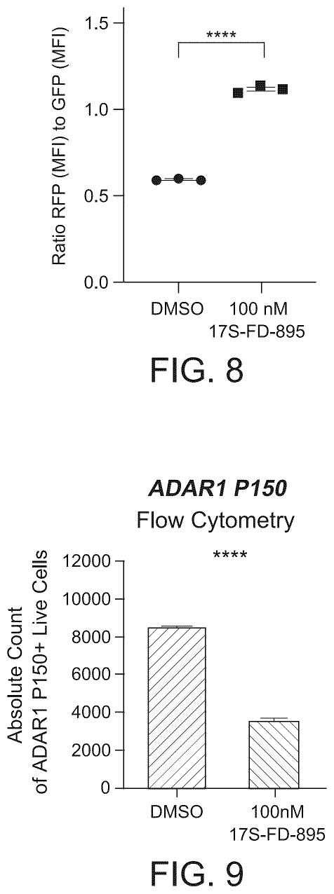

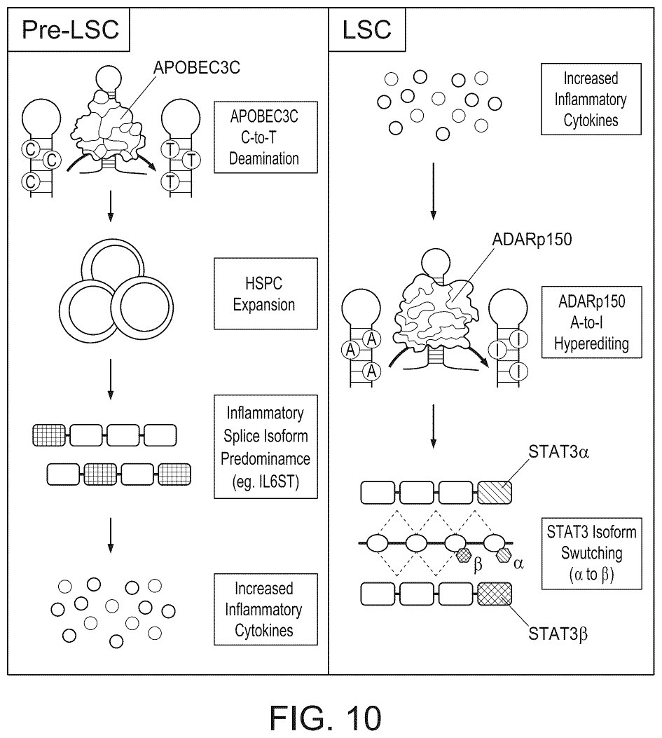

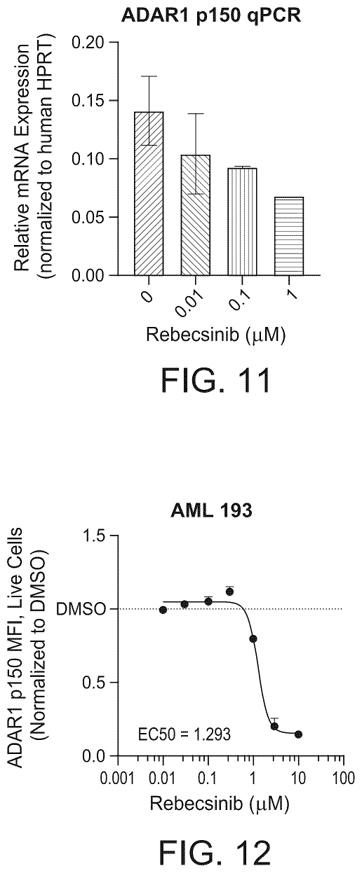

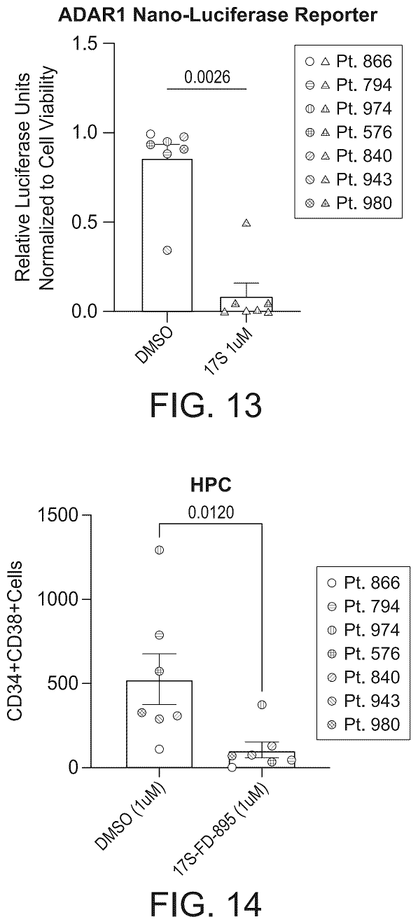

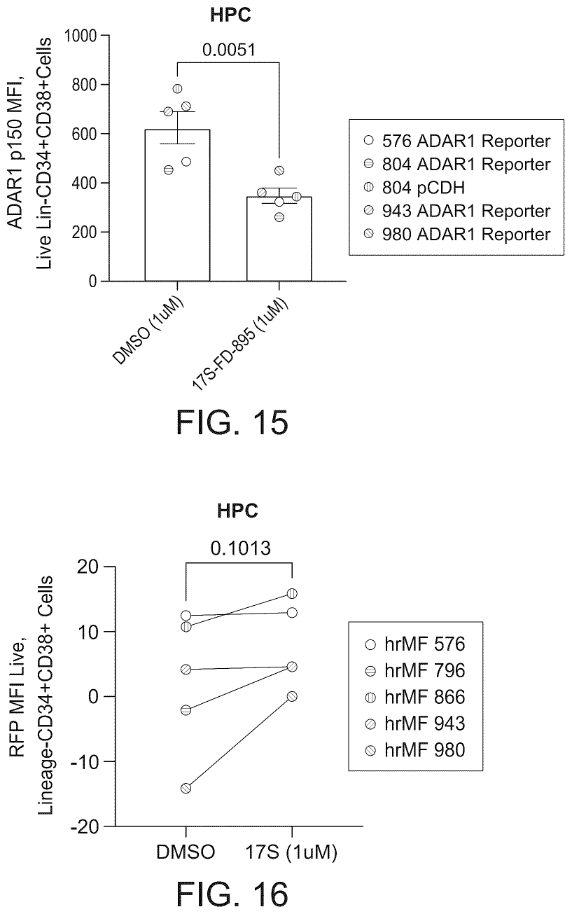

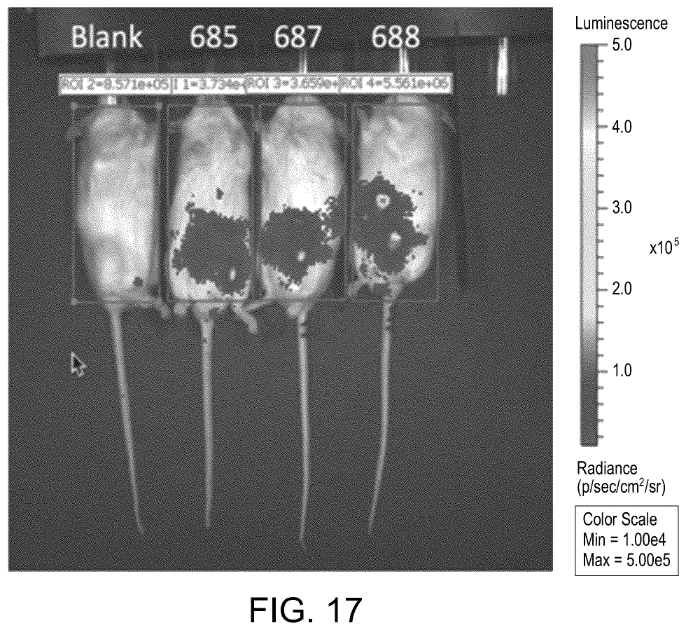

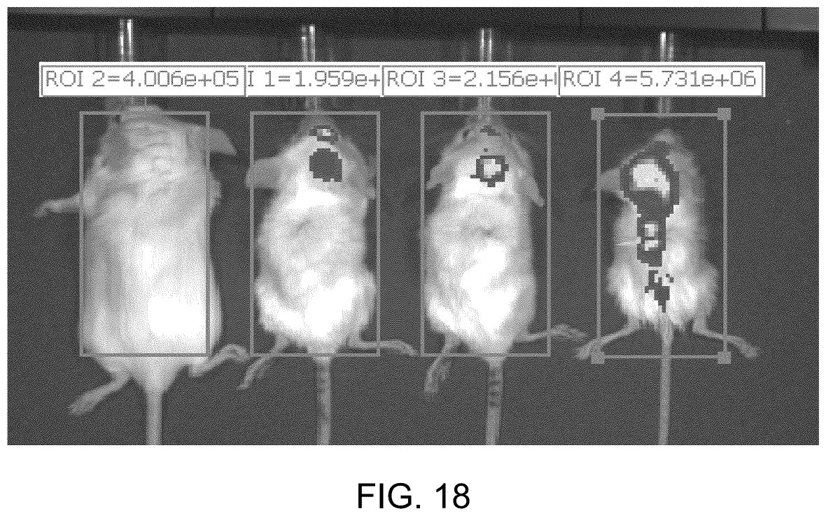

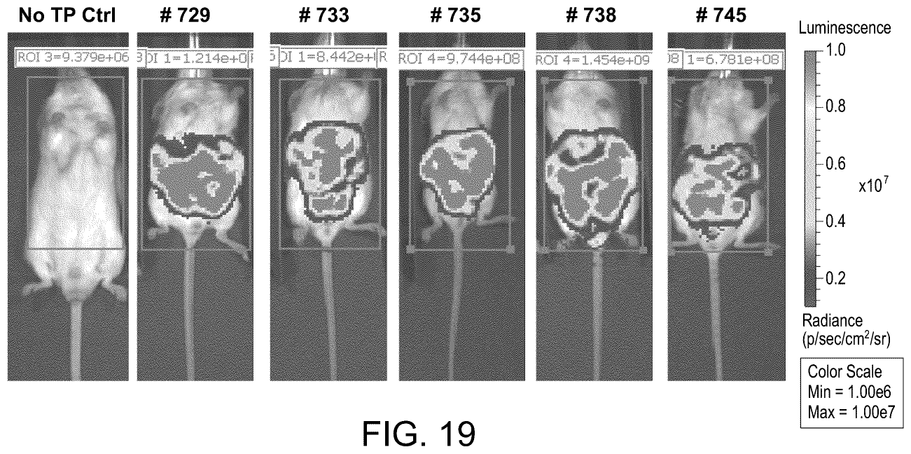

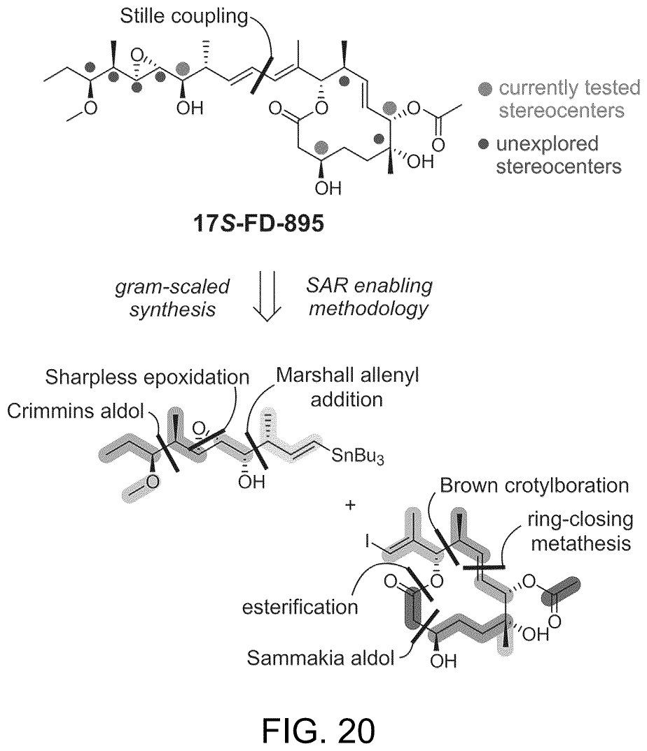

Also in this study, a patient, 68 years old, male, diagnosed at high risk of MF and AIHA, treated with ruxolitinib, having:
Mutations: MPL (47%), TET2 (42%), KMT2C (49%), SRSF2 (50%), VCUS: MPL R.V501M (49%), PAX5 (50%).
Cytogeneics: unbalanced translocation (1;6) with 1q gain, 6p loss; loss of 20q (in 4% of cells). In this study, as graphically Illustrated in FIG. 1A-C, rebecsinib (17S-FD-895) significantly inhibited CD34+Lin− cells in sAML50261 transplanted NSG-SGM3 mouse models.

### References Example 1

Crews, L.A., et al. RNA Splicing Modulation Selectively Impairs Leukemia Stem Cell Maintenance in Secondary Human AML. Cell Stem Cell 19, 599-612 (2016). Kotake, Y., et al. Splicing factor SF3b as a target of the antitumor natural product pladienolide. Nat Chem Biol 3, 570-575 (2007).
Hong, D.S., et al. A phase I, open-label, single-arm, dose-escalation study of E7107, a precursor messenger ribonucleic acid (pre-mRNA) splicesome inhibitor administered intravenously on days 1 and 8 every 21 days to patients with solid tumors. Investigational new drugs 32, 436-444 (2014).
Leon B, et al., A Challenging Pie to Splice: Drugging the Spliceosome. Angew Chem Int Ed Engl. (2017) doi: 10.1002/anie.201701065.
Kumar, D., et al. Selectivity in Small Molecule Splicing Modulation. ACS Chem Biol 11, 2716-2723 (2016).
Jiang, Q., et al. ADAR1 promotes malignant progenitor reprogramming in chronic myeloid leukemia. Proc Natl Acad Sci USA 110, 1041-1046 (2013).
Daniel Aird, et al., Sensitivity to splicing modulation of BCL2 family genes defines cancer therapeutic strategies for splicing modulators. Nature Communications volume 10, Article number: 137 (2019)
Michelle, Laetitia, et al. (2012) Proteins Associated with Exon Junction Complex also Control the Alternative Splicing of Apoptotic Regulators. Mol Cell Biology 32(5):954-67
Crews LA, et al. (2016) RNA Splicing Modulation Selectively Impairs Leukemia Stem Cell Maintenance in Secondary Human AML. Cell Stem Cell 19(5):599-612.

A number of embodiments of the invention have been described. Nevertheless, it can be understood that various modifications may be made without departing from the spirit and scope of the invention. Accordingly, other embodiments are within the scope of the following claims.

1. A pharmaceutical or therapeutic composition, a formulation or a therapeutic combination of drugs comprising:
(a) rebecsinib, also called 17S-FD-895, 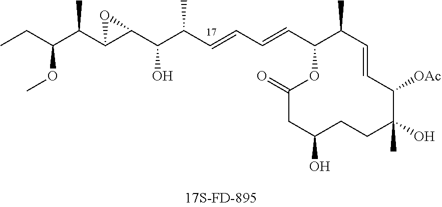 [SMILES: CC[C@H](OC)[C@@H](C)[C@H]1O[C@@H]1[C@@H](O)[C@H](C)/C=C/C=C/[C@H]1OC(=O)C[C@H](O)CC[C@@](C)(O)[C@@H](OC(C)=O)/C=C/[C@@H]1C]
or an enantiomer, stereoisomer, deuterated version, or salt thereof; and
(b) at least one second drug.

2. A method for:
treating and ameliorating a cancer;
in vivo inhibition of myeloproliferative neoplasm (MPN) or AML stem cell propagation;
the in vivo inhibition pre-leukemia stem cell (pre-LSC) transformation into leukemia stem cells (LSCs),
the in vivo inhibition of splicesomes in AML or MPN, and/or
the in vivo inhibition of transcript binding to a component of a spliceosome binding pocket or binding to ADAR1 (adenosine deaminase acting on RNA-1),
comprising:
administration to an individual in need thereof a formulation, a pharmaceutical composition or a therapeutic combination of drugs, comprising:
(a) rebecsinib, also called 17S-FD-895, 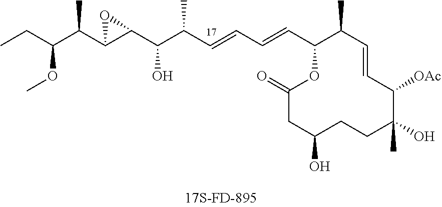 [SMILES: CC[C@H](OC)[C@@H](C)[C@H]1O[C@@H]1[C@@H](O)[C@H](C)/C=C/C=C/[C@H]1OC(=O)C[C@H](O)CC[C@@](C)(O)[C@@H](OC(C)=O)/C=C/[C@@H]1C]
or an enantiomer, stereoisomer, deuterated version, or salt thereof, or
(b) a compound of (a), or rebecsinib or 17S-FD-895, and at least one second drug.

3. The method of claim 2, wherein doses of 17S-FD-895 are administered, or formulated for administration, once a day for between one to two weeks, twice a week for 2 weeks or between about one to two weeks, followed by 2 weeks rest or 2 to 4 weeks rest, with a duration of two, three, four, five or six cycles.

4. The method of claim 2, wherein the at least one second drug comprises an ATP-competitive protein tyrosine kinase inhibitor.

5. The method of claim 2, wherein the at least one second drug comprises a JAK2 (Janus kinase 2) inhibitor.

6. The method of claim 2, wherein the at least one second drug comprises a chemotherapeutic agent.

7. The method of claim 2, wherein the at least one second drug comprises a hypomethylating agent (HMA).

8. The method of claim 2, wherein the at least one second drug comprises a second telomerase inhibitor, wherein optionally the telomerase inhibitor comprises at least one, two or three of: imetelstat, zidovudine (or azidothymidine (AZT)), stavudine (or ZERIT™), tenofovir or tenofovir disoproxil (or VIREAD™), didanosine (or VIDEX™), abacavir (ZIAGEN™), TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, ribovax, Vbx-016, ZI-HX, ZI-H04, and ZIH-03.

9. The method of claim 2, wherein the formulation, pharmaceutical composition or therapeutic combination of drugs or an active agent or drug contained therein is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen.

10. The method of claim 1, wherein an active agent or drug in the formulation, pharmaceutical composition or therapeutic combination of drugs is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more.

11. The method of claim 1, wherein an active agent or drug in the formulation, pharmaceutical composition or therapeutic combination of drugs is administered as or formulated with or formulated as an) inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially.

12. The method of claim 1, wherein the formulation, pharmaceutical composition or therapeutic combination of drugs, is or are administered to an individual in need thereof:
using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or
intravenously (IV) or intramuscularly (IM).A pharmaceutical composition comprising 17S-FD-895 (or rebecsinib), or 17S-FD-895 and at least one second drug.

13-14. (canceled)

15. The method of claim 2, wherein the cancer is a leukemia, myeloproliferative neoplasm (MPN), myelodysplastic syndrome (MDS) or multiple myeloma (MM).

16. The method of claim 15, wherein the leukemia is acute myeloid leukemia (AML), acute myeloid leukemia (AML) or chronic lymphocytic leukemia (CLL).

17. The method of claim 3: wherein doses of 17S-FD-895 are administered, or formulated for administration, once a day for between one to two weeks, twice a week for 2 weeks or between about one to two weeks, followed by 2 weeks rest or 2 to 4 weeks rest, with a duration of about four 28 day or monthly cycles.

18. The method of claim 4, wherein the ATP-competitive protein tyrosine kinase inhibitor comprises dasatinib.

19. The method of claim 5, wherein the JAK2 (Janus kinase 2) inhibitor comprises fedratinib.

20. The method of claim 19, the fedratinib is dosaged at 60 mg/kg twice daily orally, optionally for one to two or more weeks.

21. The method of claim 6, wherein the chemotherapeutic agent comprises wherein optionally the chemotherapeutic agent comprises one, two, three or more of: afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, fedratinib, floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib, vincristine, vinblastine, vinorelbine, and vindesine or eldisine.

22. The method of claim 7, wherein the HMA comprises azacitidine or decitabine.
